SEQUENCE LISTING (31 sequences)

SEQ ID NO: 1 - DNA, 33 bp, Artificial
  a primer for amplifying coding region of RepA and replication origin of pUB110
atagagctcacgaagtcgagatcagggaatgag

SEQ ID NO: 2 - DNA, 36 bp, Artificial
  a primer for amplifying coding region of RepA and replication origin of pUB110
gcgagatctctcgtcttcctaagcatccttcaatcc

SEQ ID NO: 3 - DNA, 178 bp, Artificial
  nucleotide sequence coding for replication origin of pUB110
cttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaataga

SEQ ID NO: 4 - DNA, 1005 bp, Artificial
  nucleotide sequence coding for RepA of pUB110
atgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtag

SEQ ID NO: 5 - protein, 333 aa, Artificial
  amino acid sequence coding for RepA
GVSFNIMCPNSSIYSDEKSRVLVDKTKSGKVRPWREKKIANVDYFELLHILEFKKAERVKDCAEILEYKQNRETGERKLYRVWFCKSRLCPMCNWRRAMKHGIQSQKVVAEVIKQKPTVRWLFLTLTVKNVYDGEELNKSLSDMAQGFRRMMQYKKINKNLVGFMRATEVTINNKDNSYNQHMHVLVCVEPTYFKNTENYVNQKQWIQFWKKAMKLDYDPNVKVQMIRPKNKYKSDIQSAIDETAKYPVKDTDFMTDDEEKNLKRLSDLEEGLHRKRLISYGGLLKEIHKKLNLDDTEEGDLIHTDDDEKADEDGFSIIAMWNWERKNYFIKE

SEQ ID NO: 6 - DNA, 498 bp, Artificial
  DNA sequence comprising promoter and multiple cloning site
atacccgggcatgattttaagggggttagcagatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagtttaaacctgcagagatctctcgaggcggccgcgtcgactctagacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgagctcata

SEQ ID NO: 7 - DNA, 738 bp, Artificial
  nucleotide sequence of erythromycin resistance gene
atgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 8 - DNA, 861 bp, Artificial
  nucleotide sequence of ampicillin resistance gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 9 - DNA, 615 bp, Artificial
  nucleotide sequence of replication origin of pUC19 plasmid
tcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacg

SEQ ID NO: 10 - DNA, 5014 bp, Artificial
  nucleotide sequence of plasmid pLK1-MCS
catgattttaagggggttagcagatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagtttaaacctgcagagatctctcgaggcggccgcgtcgactctagacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgagctcacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagagatcctagcagcacgccatagtgactggcgatgctgtcggaatggacgatcaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgccattctttgctgtttcgatttttatgatatggtgcaagtcagcacgaacacgaaccgtcttatctcccattatatctttttttgcactgattggtgtatcatttcgtttttctttttatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggg

SEQ ID NO: 11 - DNA, 39 bp, Artificial
  nucleotide sequence of primer using for amplication of replication origin etc. of pUC plasmid of pLK1-MCS plasmid
ctcaggcctgtgagttttcgttccactgagcgtcagacc

SEQ ID NO: 12 - DNA, 36 bp, Artificial
  nucleotide sequence of primer using for amplication of replication origin etc. of pUC plasmid of pLK1-MCS plasmid
gcgggatccctcgtcttcctaagcatccttcaatcc

SEQ ID NO: 13 - DNA, 208 bp, Artificial
  nucleotide sequence of thiolase promoter region
catgattttaagggggttagcagatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaattt

SEQ ID NO: 14 - DNA, 4060 bp, Artificial
  nucleotide sequence of DNA fragment amplified of coding region of RepA and replication origin of pUB110, MCS, thiolase promoter region and replication origin region of pUC19 of pLK1-MCS plasmid
aggcctatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagagatcctagcagcacgccatagtgactggcgatgctgtcggaatggacgatcaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgccattctttgctgtttcgatttttatgatatggtgcaagtcagcacgaacacgaaccgtcttatctcccattatatctttttttgcactgattggtgtatcatttcgtttttctttttatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgggatcc

SEQ ID NO: 15 - DNA, 32 bp, Artificial
  nucleotide sequence of primer used for amplication of chloramphenicol resistance gene comprised in pGS1-MCS plasmid
ataagatctcgactttttaacaaaatatattg

SEQ ID NO: 16 - DNA, 36 bp, Artificial
  nucleotide sequence of primer used for amplication of chloramphenicol resistance gene comprised in pGS1-MCS plasmid
atacccgggcagctctaggcaatattatatctgcaa

SEQ ID NO: 17 - DNA, 902 bp, Artificial
  nucleotide sequence of DNA fragment amplifying chloramphenicol resistance gene comprised in pGS1-MCS plasmid
agatctcgactttttaacaaaatatattgataaaaataataatagtgggtataattaagttgttagagaaaacgtataaattagggataaactatggaacttatgaaatagattgaaatggtttatctgttaccccgtaggatccacttgaatttaaaaggagggaacttagatggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacagaattatactccctttggcaattcaagttcatcacgcagtatgtgacggatttcacatttgccgttttgtaaacgaattgcaggaattgataaatagttaacttcaggtttgtctgtaactggcgccacttaatgatttgccagtaaaagagattgtttctagctctcacattcttgcagatataatattgcctagagctgcccggg

SEQ ID NO: 18 - DNA, 146 bp, Artificial
  DNA sequence comprising trascription termination factor of acetoacetate decarboxylase gene
gaattctaaaaataagagttaccttaaatggtaactcttatttttttaatattgttactggccgtcgttttacaacgtcgtgactgggaaaaccctggcgttacccaacttaatcgccttgcagcacatccccctttcgccagctg

SEQ ID NO: 19 - DNA, 3660 bp, Artificial
  nucleotide sequence of pCK2-MCS shuttle plasmid
catgattttaagggggttagcagatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagtttaaacctgcagagatctctcgaggcggccgcgtcgactctagacccgggaattctaaaaataagagttaccttaaatggtaactcttatttttttaatattgttactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgagctcacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagggatctcgactttttaacaaaatatattgataaaaataataatagtgggtataattaagttgttagagaaaacgtataaattagggataaactatggaacttatgaaatagattgaaatggtttatctgttaccccgtaggatccacttgaatttaaaaggagggaacttagatggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacagaattatactccctttggcaattcaagttcatcacgcagtatgtgacggatttcacatttgccgttttgtaaacgaattgcaggaattgataaatagttaacttcaggtttgtctgtaactggcgccacttaatgatttgccagtaaaagagattgtttctagctctcacattcttgcagatataatattgcctagagctgccccctgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggg

SEQ ID NO: 20 - DNA, 2589 bp, Artificial
  nucleotide sequence of AdhE1 gene of Clostridium acetobutylicum
atgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaa

SEQ ID NO: 21 - DNA, 41 bp, Artificial
  nucleotide sequence of primer used for amplication of AdhE1 gene of Clostridium acetobutylicum
cacgtttaaacatgaaagtcacaacagtaaaggaattagat

SEQ ID NO: 22 - DNA, 42 bp, Artificial
  nucleotide sequence of primer used for amplication of AdhE1 gene of Clostridium acetobutylicum
cacactcgagttaaggttgttttttaaaacaatttatataca

SEQ ID NO: 23 - DNA, 289 bp, Artificial
  nucleotide sequence of thiolase promoter of Clostridium acetobutylicum
ttagaatgaagtttcttatgcacaagtattttttattacattaatatagttaaaatataaacttatgtatttatgctaaaacatgattttaagggggttagcatatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaattt

SEQ ID NO: 24 - DNA, 45 bp, Artificial
  nucleotide sequence a primer used for amplication of thiolase promoter of Clostridium acetobutylicum
cacactcgagttagaatgaagtttcttatgcacaagtatttttta

SEQ ID NO: 25 - DNA, 40 bp, Artificial
  nucleotide sequence a primer used for amplication of thiolase promoter of Clostridium acetobutylicum
tctagatcttaacctcctaaattttgatacggggtaacag

SEQ ID NO: 26 - DNA, 1324 bp, Artificial
  nucleotide sequence of a CtfAB gene group
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataaaatgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttaa

SEQ ID NO: 27 - DNA, 44 bp, Artificial
  nucleotide sequence of a primer used in amplication of a CtfAB gene group
cacagatctatgaactctaaaataattagatttgaaaatttaag

SEQ ID NO: 28 - DNA, 44 bp, Artificial
  nucleotide sequence of a primer used in amplication of a CtfAB gene group
cacgaattcttaaacagccatgggtctaagttcattggatatga

SEQ ID NO: 29 - DNA, 3773 bp, Artificial
  nucleotide sequence of a operon comprising Xylose kinase and Xylulose isomerase genes of Clostridium acetobutylicum
atgaggtatttattaggtatagacgttggaacatcaggaaccaagacagctttgtttgatgaatgtggaaatacaataaaaacttcaacgcatgagtatgagttatttcaaccacaggttggatgggcagaacaaaatcctgaaaattggtggacagcttgtgttaaaggtataagagaagtaattgaaaaaagtaaaattgatcctttagatattaaaggaattggtataagtggacaaatgcatggacttgttttgattgacaaagagtacaaagtaattagaaactccataatttggtgtgatcagagaacagaaaaggaatgtactcagattacagataccataggaaaagaaaaattaattaggataacaggcaatcctgcattaacgggtttcaccctttcaaaactattatgggttaggaataatgaaccagacaattataagagaatatataaggttcttcttcctaaggattatataagatttaaacttactggagtttttgctgccgaagtttcagatgctagtggtactcagatgttagatataaatactagaaattggagtgaagaacttttagatgacttaagaatagataagaacatattacctgatgtatatgaatcagttgttgttagtggatgcgttatagaaaaagcttcaaaagagactaagttagcagttaacactcccgttgtaggtggggcaggagatcaagcagctggagctatagggaatggaatcgttagagaaggtttaatatcaacagtgataggaacttctggagtagtatttgcggctacagatacgcctagatttgatagtaagggaagagttcatactctttgtcatgcagtgcctaataagtggcatataatgggagttactcagggagcaggcttatcattaaattggtttaaaaggacattttgtgcaaaagaaattttagaaagtaaagaggctggaattaatatttatgatttattgaccgaaaaagcatcacaatcaaagcctggttctaatgggataatttatttgccataccttatgggtgaaagaacaccgcacatagatccaaatgtaaaaggagcttttttaggtatatcacttataaataaccacaatgattttgtgcgtagtatattagaaggagtaggctttagtttaaagaattgtctcgatattattgagaatatgaaggttaatattgaggaaatacgagtaagtggtggaggtgcagaaagcagcatatggagacaaatattgtcagatatatttaactatgagcttacaacagtaaaggcatcagaaggaccagcacttggcgtagcaatacttgcaggagttggtgcaggaatatataattccgtggaagaggcttgtgacaaaatagtaaaaggaaacgaaaaggttatgccaaatgcaaatttaatagaagtatattcaaaggtatatgaggtatataattcagcttatcctaaaataaaagatatataaatagttaaaagtaggagcgcttttatttaaagagtagattatctaaaaaacttattaaacaaactttaaaaacatatatttattagagaagtttaaatcatattggtaagatattttttaaataaaaccaatttaaattatatggggagtgtgagaatatggtaattaaaagtgttacagacaaggagttcaaaaaatatggcaagatattaagagattatgattgtacagagataatagaaaaaatgaaatctacaccacttccaggtgatgtagtatatgaaccatcaattaaagaacttgaagagcttgatattgcaaaggaattaaaggaaagagaatatggagagcttccaatacaagtaggatactgcaatggtaacaattatttattaaatgcagtcgaatatcatcgttcatctgaaataaatatagcagtaacagatttcatactacttttaggctctagagatgatattgaagaagattactcatatagtacttctaaaattaaagcatttaaagttccagctggtactgctattgaggtttatgcaactactcttcactatgcaccttgcaatgtaaatgaagggggattcagatgtgttgtggttttgccaagagatacaaatttgcctttagaaaaagagtataaaaaaactggtgaagatgctctactttttgctaaaaacaaatggttaattgcccataaggatactaacctagataaagaaggagcatttataggattaataggtaaaaatatttcaataaaataaaatggaggaattaaaatgaataatacaccaaaattaaaattaggaattgttgcagtaagtagagactgctttccaatggaattatcagagaatagaagaaaggcagtagtagatgcttacaatggagaaatcttcgagtgcttaacaactgttgaaaatgaaaaagatatgagaaaagctttaaaagaagtgaaaagtgcaggtgtaaatgcactagtagtatatttaggtaactttggaccagaaactccagaaactttaattgctaaagaatttgatggaccagttatgtttgcagccgcagcagaagagagaggagacaatctagttaatggacgtggagatgcttattgtggaatgctaaatgcaagctataatttagcacttagaaacataaatgcttatattcctgaatatccagtgggaacagcttcagatgttgcaaatatgattaatgaatttgtaccggttgctactgcattaattggattaaagaatttaaagattatttcttttgggccaagacctcaggatttcctagcttgcaatgctcctattaagcagttatataatttaggtgttgaaatagaggaaaattcggagcttgatttgtttgcctcatttaatgaacatgaaaacgattctagaattccagatgtagtaaaagacatggaggaagagttaggtgagggcaataaaatgcctggtattttgccaaagcttgctcaatatgaacttacattactagattgggcagaggagcataaaggatcaagagaatatgttgtgtttgcaaataaatgctggccagcgtttcaaacacaatttggatgtgtgccttgctatgtaaacagtagactaacggctagaggaattccagtatcttgtgaggttgacatttatggagctttaagtgaatacattggaacatgtgtaagtcaagatgttgtaaccttacttgatattaataacacagtaccaaaagatatgtatgaatcagaaattaaaagcaaatttaactatacgttaaaggataccttcatgggttttcactgtggaaacacagcagcatgtaaattaacaagcggaactatgaaaaatcaaatgattatggcaagagctcttgagccaaatcaagagcctaatataactagaggaacgttagagggagatattgtaccaggagaaatcactttcttccgtctacaaagtaatgcagatagtgagttaacagcctatgtagcagaaggagaggttttacctgttaagacacgctcttttggatctattggagtatttgcaattcctcaaatggggagattttaccgccatgtactaattgaaaatagattcccacaccatggtgcagttgcatttggacattttggtaaagcaatttataatttgtttagatatttgggagttaaagaggtaggttttaataggccaaaggaaatgctatataaaacagaaaatccttttgagtaa

SEQ ID NO: 30 - DNA, 36 bp, Artificial
  nucleotide sequence of a primer used for amplication of a operon comprising Xylose kinase and Xylulose isomerase genes of Clostridium acetobutylicum
cgctgcagatgaggtatttattaggtatagacgttg

SEQ ID NO: 31 - DNA, 40 bp, Artificial
  nucleotide sequence of a primer used for amplication of a operon comprising Xylose kinase and Xylulose isomerase genes of Clostridium acetobutylicum
ggcggccgcttactcaaaaggattttctgttttatatagc

(19) United States
(12) Patent Application Publication
(10) Pub. No.: US 2023/0183715 A1
(43) Pub. Date: Jun. 15, 2023

(54) SHUTTLE PLASMID REPLICABLE IN CLOSTRIDIUM AND E. COLI AND RECOMBINANT MICROORGANISM PREPARED THEREWITH AND HAVING ENHANCED PENTOSE METABOLISM AND FERMENTATION PERFORMANCE

(71) Applicant: GS CALTEX CORPORATION, Seoul (KR)

(72) Inventor: Sun-Hwa CHOI, Sejong (KR)

(21) Appl. No.: 16/767,931

(22) PCT Filed: Nov. 28, 2018
(86) PCT No.: PCT/KR2018/014875

§ 371 Date: May 28, 2020

(30) Foreign Application Priority Data
Nov. 28, 2017 (KR) 10-2017-0160014

(51) Int. Cl. C12N 15/66; C12N 15/65; C12N 15/70; C12N 15/74; C12N 9/12

(52) U.S. Cl. CPC C12N 15/66; C12N 15/65; C12N 15/70; C12N 15/74; C12N 9/12

(57) ABSTRACT

The present invention relates to a shuttle plasmid replicable in Clostridium and E. coli, the shuttle plasmid comprising: a nucleotide sequence of the first replication origin allowing replication in E. coli; a nucleotide sequence coding for a replication protein region derived from pUB110 plasmid; and an expression terminator sequence of a gene.

### FIELD

The present disclosure relates to a shuttle plasmid replicable in Clostridium and Escherichia coli, and a recombinant microorganism produced using the shuttle plasmid.

### DESCRIPTION OF RELATED ART

Butanol is an intermediate compound having a wide range of applications, for example, for cosmetics, perfumes, hormones, hygiene, industrial coatings, paint additives, textiles, plastic monomers, medical supplies, vitamins, antibiotics, and pesticides, and thus has great usefulness.

A conventional butanol production method in which a sugar is fermented using Clostridium strain, thereby producing butanol, acetone and ethanol was used until the 80 s. Since then, an oxo process for synthesizing butanol from propylene obtained from petroleum has been widely used. However, the petroleum-based butanol production method uses high temperature and high pressure, which makes the process complicated, and emits a large amount of hazardous waste and carbon dioxide. Recently, there has been an increasing demand for environmentally friendly production of butanol via fermentation of microorganisms from renewable resources.

However, in order to produce butanol at an industrially useful scale using microorganisms, a selectivity, yield and productivity of butanol, that is, a production amount of butanol per unit time must be excellent. However, none of wild-type and recombinant microorganisms for bio-butanol production which have been known until now has not satisfied the above requirements.

Specifically, for example, it is known that a wild type Clostridium acetobutylicum ATCC824 strain produces acetone, ethanol, and butanol at a mass ratio of about 3:1:6 via fermentation, and produces small amounts of acetic acid and butyric acid. In this connection, a yield of the wild type strain is about 25%, and a final concentration is about 10 g/L. Microorganisms having an acetyl coenzyme A biosynthetic pathway and a butyryl coenzyme A biosynthetic pathway such as the Clostridium acetobutylicum are generally known to synthesize acetone, butanol and ethanol. Recently, efforts have been made to produce butanol more efficiently due to development of metabolic engineering technology. In particular, a genomic sequence of the Clostridium acetobutylicum has recently been known, and thus research on metabolic pathway manipulation thereof has been actively conducted.

For example, a result of overexpressing adhE1 and ctfAB genes simultaneously in Clostridium acetobutylicum M5 strain with deletion of a large plasmid in which butanol-generation related genes (adc, ctfAB and adhE1 (alcohol/aldehyde dehydrogenase) and adhE2 (alcohol/aldehyde dehydrogenase)) are present has been reported. According to this report, butanol selectivity was improved to 0.78 based on a mass ratio, but growth of the strain was inhibited and acetic acid production increased, so that productivity and yield of the butanol were significantly reduced (Lee, et al., Biotechnology Journal, 4: 1432-1440, 2009; Lee, et al., WO 2009/082148).

It has been reported that both of when a pta gene that converts acetyl coenzyme A (Acetyl-CoA) to acetate is deleted, and when both the pta gene and a buk gene that converts butyryl coenzyme A (butyryl-CoA) to butyrate are deleted and aad (alcohol/aldehyde dehydrogenase) is overexpressed, butanol concentration, selectivity and yield increased. However, the both cases had limitations in terms of butanol productivity and strain stability (LEE et al., WO 2011/037415). Further, butanol productivity was still low when a CtfB gene for encoding coenzyme A transferase (CoAT) was additionally deleted in a mutant strain in which the pta and buk genes were deleted (LEE et al., WO 2011/037415).

In addition, one report has disclosed that, in fermentation using a mutant Clostridium beijerinckii BA101 strain induced via random mutation, and using maltodextrin as a carbon source, 18.6 g/l butanol was produced (Ezeji et al., Appl. Microbiol. Biotechnol., 63: 653, 2004). However, even when the recombinant strains are used, the productivity of butanol as a final product is low, thus making industrial use impossible.

Further, a report has been made that deleting a ctfAB encoding the coenzyme A transferase or adc as acetoacetic acid decarboxylase may reduce concentration of acetone and increase the selectivity of butanol. However, a final concentration of butanol was lower than 10 g/L and the stability of strain was problematic (Jiang et al., Metab. Eng., 11 (4-5): 284-291, 2009).

Further, when the adc (acetoacetic acid decarboxylase) and ctfAB (encoding coenzyme A transferase) genes were overexpressed in the wild-type Clostridium acetobutylicum, productivities of acetone, ethanol, and butanol increased by 95%, 90%, and 37%, respectively, compared to the wild-type. However, butanol selectivity and yield were low (Mermelstein et al., Biotechnol. Bioeng., 42: 1053, 1993).

Therefore, for industrial scale production of butanol, it is necessary to develop a Clostridium strain with effective high metabolic activity. Further, in order to develop Clostridium strain with high metabolic activity, it is desired to develop an effective host/plasmid system.

Therefore, an improved shuttle vector such as a shuttle plasmid replicable in Clostridium and Escherichia coli has been developed (Korean Patent No. 10-1565726). The present inventors have further developed the shuttle plasmid into a shuttle plasmid which has a reduced size, is replicable in Clostridium and Escherichia coli using only one antibiotic resistant gene (Chloramphenicol resistant gene) without replacing the antibiotic resistant gene, and has better replication stability. Further, the present inventors have used the shuttle plasmid according to the present disclosure, thereby producing a recombinant microorganism. We have identified excellent butanol productivity by the recombinant microorganism. In this way, the present disclosure has been completed.

### PRIOR ART DOCUMENTS

(Patent Document 1) Korean Patent No. 10-1565726
(Patent Document 2) Korean Patent No. 10-1548480
(Patent Document 3) Korean Patent No. 10-1487057

### DISCLOSURE

### Technical Purposes

A purpose of the present disclosure is to provide a shuttle plasmid replicable in Clostridium and Escherichia coli with high replication safety and simplicity.

Another purpose of the present disclosure is to provide a recombinant microorganism capable of exhibiting high butanol selectivity and yield and low ethanol selectivity and thus of continuously producing bio-butanol at an industrial scale.

Still another purpose of the present disclosure is to provide a recombinant microorganism having maintained or improved fermentation performance, and having increased consumption efficiency of pentose, such as xylose.

### Technical Solutions

To achieve the purposes, the present disclosure provides a shuttle plasmid replicable in Clostridium and Escherichia coli,

wherein the shuttle plasmid includes:

a nucleic acid sequence of a first replication origin replicable in Escherichia coli;

a nucleic acid sequence for encoding a replication protein region derived from pUB110 plasmid; and

an expression terminator sequence of a gene.

Further, the present disclosure provides a recombinant microorganism produced using the shuttle plasmid.

Further, the present disclosure provides a method for producing a recombinant microorganism using the shuttle plasmid.

### Technical Effects

The shuttle plasmid according to the present disclosure may be replicable in Clostridium and Escherichia coli with high replication safety and convenience.

Further, the recombinant microorganism according to the present disclosure has high ABE (acetone, ethanol, and butanol) yield, high butanol productivity, high butanol selectivity, and low ethanol selectivity. Therefore, the recombinant microorganism according to the present disclosure may continuously produce biobutanol at an industrial scale.

### BRIEF DESCRIPTIONS OF DRAWINGS

FIG. 1 is a gene map of Staphylococcus aureus-derived cryptic plasmid pUB110.

FIG. 2 shows a preparation process of a shuttle plasmid pLK1-MCS from pUB110 cryptic plasmid and pMTL500E plasmid having a nucleic acid sequence of a replication origin of pUC19 plasmid.

FIG. 3 is a schematic diagram showing a preparation process of pCK2-MCS shuttle plasmid using pLK1-MCS plasmid and pGS1-MCS plasmid.

FIG. 4 shows a pLK1-MCS plasmid.

FIG. 5 shows a pGS1-MCS plasmid.

FIG. 6 shows a pCK2-MCS shuttle plasmid.

### DETAILED DESCRIPTIONS

Entire disclosures of Korean Patent No. 10-1565726 and Korean Patent No. 10-1548480 are incorporated herein by reference.

The present disclosure provides a shuttle plasmid replicable in Clostridium and Escherichia coli,

wherein the shuttle plasmid includes:

a nucleic acid sequence of a first replication origin replicable in Escherichia coli;

a nucleic acid sequence for encoding a replication protein region derived from pUB110 plasmid; and

an expression terminator sequence of a gene.

Further, the present disclosure provides a method for producing a recombinant microorganism, wherein the method includes:

preparing a shuttle plasmid according to the present disclosure;

introducing at least one gene into the shuttle plasmid, thereby producing a first recombinant shuttle plasmid; and

introducing the first recombinant shuttle plasmid into a microorganism.

Further, the present disclosure provides a production method of a recombinant microorganism,

wherein the method includes:

introducing a second recombinant shuttle plasmid into a microorganism; and

further introducing a first recombinant shuttle plasmid into the microorganism, wherein the first recombinant shuttle plasmid is produced by introducing at least one gene into the shuttle plasmid according to the present disclosure.

Further, the present disclosure provides a production method of a recombinant microorganism,

wherein the method includes:

preparing a shuttle plasmid according to the present disclosure;

introducing at least one gene into the shuttle plasmid, thereby producing a first recombinant shuttle plasmid; and

simultaneously introducing the first recombinant shuttle plasmid and a second recombinant shuttle plasmid into a microorganism.

Further, the present disclosure relates to a method for obtaining a fermentation product,

wherein the method includes:

culturing the recombinant microorganism produced by the production method according to the present disclosure, thereby producing a culture; and

obtaining a fermentation product from the culture.

Hereinafter, the present disclosure will be described in detail.

Shuttle plasmid according to the present disclosure

The shuttle plasmid according to the present disclosure is replicable in Clostridium and Escherichia coli, and includes: a nucleic acid sequence of a first replication origin replicable in Escherichia coli; a nucleic acid sequence for encoding a replication protein region derived from pUB110 plasmid; and an expression terminator sequence of a gene.

A nucleic acid sequence of a first replication origin that is replicable in the Escherichia coli may be a nucleic acid sequence of a replication origin of pUC19 plasmid, and includes a nucleic acid sequence having 80% or more identity with the nucleic acid sequence of the replication origin of pUC19 plasmid as long as it functions as a replication origin. Preferably, the nucleic acid sequence of the first replication origin that is replicable in the Escherichia coli may be a nucleic acid sequence of SEQ ID NO: 9, and includes a nucleic acid sequence having 80% or more identity with the nucleic acid sequence of SEQ ID NO: 9 as long as it functions as a replication origin that is replicable in Escherichia coli.

A nucleic acid sequence for encoding a replication protein region derived from a pUB110 plasmid may be a nucleic acid sequence for encoding a replication protein (RepA) of the pUB110 plasmid and includes a nucleic acid sequence having 80% or more identity therewith. Preferably, the nucleic acid sequence for encoding the replication protein region derived from the pUB110 plasmid may be a nucleic acid sequence of SEQ ID NO: 4, and includes a nucleic acid sequence having 80% or more identity therewith. Further, the nucleic acid sequence for encoding the replication protein region derived from the pUB110 plasmid may be a nucleic acid sequence for encoding an amino acid sequence of SEQ ID NO: 5, and includes a nucleic acid sequence having 80% or more identity therewith.

The expression terminator sequence of the gene may be a nucleic acid sequence of a transcription terminator (Adc terminator) of a acetoacetate decarboxylase gene of Clostridium acetobutylicum, and includes a sequence having 80% or more identity therewith as long as it maintains gene expression termination function. Preferably, the expression terminator sequence of the gene may be a nucleic acid sequence of SEQ ID NO: 18, and may include a sequence having 80% or more identity therewith as long as it maintains the gene expression termination function.

Further, the shuttle plasmid according to the present disclosure may additionally include an antibiotic resistant gene expressed in Clostridium and Escherichia coli. The antibiotic resistant gene is preferably a chloramphenicol resistant gene. More preferably, the antibiotic resistant gene has a nucleic acid sequence of SEQ ID NO: 17.

Thus, the shuttle plasmid according to the present disclosure is replicable in both Clostridium and Escherichia coli without replacing the antibiotic resistant gene. That is, in a conventional shuttle plasmid, such as pLK1-MCS shuttle plasmid, when introducing the shuttle plasmid into the Clostridium and then introducing the shuttle plasmid into the Escherichia coli, or when introducing the shuttle plasmid into the Escherichia coli and then introducing the shuttle plasmid into the Clostridium, the antibiotic resistant gene must be replaced. However, in the shuttle plasmid according to the present disclosure, it is not necessary to replace the antibiotic resistant gene when introducing the shuttle plasmid into Clostridium and, then, introducing the shuttle plasmid into Escherichia coli. In another example, in the shuttle plasmid according to the present disclosure, it is not necessary to replace the antibiotic resistant gene when introducing the shuttle plasmid into Escherichia coli and, thereafter, introducing the shuttle plasmid into the Clostridium.

The shuttle plasmid according to the present disclosure has a size of 3000 bp to 4000 bp, preferably, a size of 3200 bp to 3800 bp.

The shuttle plasmid according to the present disclosure may further include a nucleic acid sequence of a thiolase promoter region, a nucleic acid sequence of a multiple cloning site (MCS), or a nucleic acid sequence of a replication origin of pUB110 plasmid. Preferably, the shuttle plasmid according to the present disclosure may further include a following i) or a following ii): i) nucleic acid sequences of a thiolase promoter region and a multiple cloning site (MCS), ii) a nucleic acid sequence of a replication origin of pUB110 plasmid. More preferably, the shuttle plasmid according to the present disclosure may further include a nucleic acid sequence of a thiolase promoter region, a nucleic acid sequence of a multiple cloning site (MCS) site, and a nucleic acid sequence of a replication origin of the pUB110 plasmid. The nucleic acid sequence of the thiolase promoter region and the multiple cloning site (MCS) may be a nucleic acid sequence of SEQ ID NO: 6, and may include a sequence with 80% or more identity therewith, as long as it maintains a promoter function and a multiple cloning function. The nucleic acid sequence of the replication origin of the pUB110 plasmid may be a nucleic acid sequence of SEQ ID NO: 3, and may include a sequence having 80% or more identity therewith as long as it acts as a replication origin.

### Production Method of Recombinant Microorganism

The present disclosure provides a production method of a recombinant microorganism, the method including preparing the shuttle plasmid according to the present disclosure; introducing at least one gene into the shuttle plasmid, thereby producing a first recombinant shuttle plasmid; and introducing the first recombinant shuttle plasmid into microorganisms. In this connection, the method may further include, after introducing the first recombinant shuttle plasmid into the microorganisms, introducing a second recombinant shuttle plasmid into the microorganisms.

Further, the present disclosure provides a production method of a recombinant microorganism, the method including introducing a second recombinant shuttle plasmid into microorganisms; and further introducing a first recombinant shuttle plasmid into the microorganism, wherein the first recombinant shuttle plasmid is produced by introducing at least one gene into the shuttle plasmid according to the present disclosure.

Further, the present disclosure provides a production method of a recombinant microorganism, wherein the method includes: preparing a shuttle plasmid according to the present disclosure; introducing at least one gene into the shuttle plasmid, thereby producing a first recombinant shuttle plasmid; and simultaneously introducing the first recombinant shuttle plasmid and a second recombinant shuttle plasmid into a microorganism.

That is, the recombinant microorganism according to the present disclosure may be produced by sequentially or simultaneously introducing the first recombinant shuttle plasmid and the second recombinant shuttle plasmid into microorganisms. In the sequential introducing, the first recombinant shuttle plasmid may be first introduced, and, then, the second recombinant shuttle plasmid may be introduced, or vice versa.

In this connection, the second recombinant shuttle plasmid is preferably a recombinant shuttle plasmid having the same replication origin as a replication origin of the shuttle plasmid according to the present disclosure. More preferably, the second recombinant shuttle plasmid is a recombinant shuttle plasmid obtained by introducing a gene into the pLK1-MCS shuttle plasmid.

The gene introduced into the shuttle plasmid, that is, the gene introduced into the first recombinant shuttle plasmid may be at least one selected from a group consisting of a gene for encoding xylose kinase, a gene for encoding xylulose isomerase, a gene for encoding alcohol/aldehyde dehydrogenase, and a gene for encoding coenzyme A transferase.

In this connection, the second recombinant shuttle plasmid may include a gene different from the gene introduced into the first recombinant shuttle plasmid. Alternatively, the second recombinant shuttle plasmid may include the same gene as the gene introduced into the first recombinant shuttle plasmid. Further, the second recombinant shuttle plasmid may include at least one gene selected from a group consisting of a gene for encoding xylose kinase, a gene for encoding xylulose isomerase, a gene for encoding alcohol/aldehyde dehydrogenase, and a gene for encoding coenzyme A transferase.

That is, the second recombinant shuttle plasmid may include at least one gene selected from a group consisting of a gene for encoding xylose kinase, a gene for encoding xylulose isomerase, a gene for encoding alcohol/aldehyde dehydrogenase, and a gene for encoding coenzyme A transferase. In this connection, the second recombinant shuttle plasmid may include the same or different gene as or from the gene introduced into the first recombinant shuttle plasmid.

The at least one gene may be a gene for encoding xylose kinase or a gene for encoding xylulose isomerase. Preferably, the at least one gene may be a gene for encoding xylose kinase and a gene for encoding xylulose isomerase. The gene for encoding the xylose kinase may be XylB, and the gene for encoding the xylulose isomerase may be XylA. The at least one gene may be XylBA operon. Preferably, the at least one gene may be XylBA operon of SEQ ID NO: 29.

In this connection, the microorganism may be a microorganism having an acetyl coenzyme A biosynthetic pathway and a butyryl coenzyme A biosynthetic pathway. The microorganism may be a microorganism including a gene for encoding alcohol/aldehyde dehydrogenase and a gene for encoding coenzyme A transferase. The microorganism may preferably be Escherichia coli or Clostridium. The gene for encoding the alcohol/aldehyde dehydrogenase may be adhE I, and may preferably be a gene of SEQ ID NO: 20. The gene for encoding the coenzyme A transferase may be ctfAB, and may preferably be a gene of SEQ ID NO: 26.

Further, the present disclosure provides a method for producing a recombinant microorganism having improved butanol production capacity, the method including introducing a gene for encoding xylose kinase or a gene for encoding xylulose isomerase into shuttle plasmid according to the present disclosure; and introducing a first recombinant shuttle plasmid into which the gene has been introduced, into microorganisms having an acetyl coenzyme A biosynthetic pathway and a butyryl coenzyme A biosynthetic pathway.

Microorganism

A gene may be introduced into microorganisms using the shuttle plasmid according to the present disclosure, thereby producing recombinant microorganisms. In this connection, the microorganism may be a microorganism having an acetyl coenzyme A biosynthetic pathway and a butyryl coenzyme A biosynthetic pathway. Alternatively, the microorganism may be a microorganism including a gene for encoding alcohol/aldehyde dehydrogenase and a gene for encoding coenzyme A transferase. The microorganism may preferably be Escherichia coli or Clostridium.

Recombinant Microorganism

The present disclosure relates to recombinant microorganisms produced according to the production method of the recombinant microorganisms according to the present disclosure. Further, the present disclosure relates to recombinant microorganisms for butanol production, the recombinant microorganisms including a gene for encoding xylose kinase and/or a gene for encoding xylulose isomerase. The recombinant microorganism for producing butanol, including the gene for encoding the xylose kinase and/or the gene for encoding xylulose isomerase may further include a gene for encoding alcohol/aldehyde dehydrogenase and a gene for encoding coenzyme A transferase. In this connection, the recombinant microorganism may have an acetyl coenzyme A biosynthetic pathway and a butyryl coenzyme A biosynthetic pathway. Preferably, the recombinant microorganism is recombinant Clostridium. More preferably, the recombinant microorganism is recombinant Clostridium acetobutylicum.

Further, the present disclosure relates to recombinant microorganisms for butanol production, the recombinant microorganisms being produced by introducing, into microorganisms having an acetyl coenzyme A biosynthetic pathway and a butyryl coenzyme A biosynthetic pathway, at least one, preferably, at least two, more preferably, at least three, selected from a group consisting of a gene for encoding xylose kinase, a gene for encoding xylulose isomerase, a gene for encoding alcohol/aldehyde dehydrogenase, and a gene for encoding coenzyme A transferase. The gene may be introduced into the microorganism using the shuttle plasmid according to the present disclosure. Preferably, the recombinant microorganism according to the present disclosure may be produced by introducing the genes into microorganisms using: a first recombinant shuttle plasmid produced by introducing at least one selected from a group consisting of a gene for encoding xylose kinase, a gene for encoding xylulose isomerase, a gene for encoding alcohol/aldehyde dehydrogenase, and a gene for encoding coenzyme A transferase into the shuttle plasmid according to the present disclosure; and a second recombinant shuttle plasmid including at least one gene selected from a group consisting of a gene for encoding xylose kinase, a gene for encoding xylulose isomerase, a gene for encoding alcohol/aldehyde dehydrogenase, and a gene for encoding coenzyme A transferase. The recombinant microorganism for butanol production may be produced by introducing, into microorganisms, at least one, preferably, at least two, more preferably, at least three, selected from a group consisting of a nucleic acid sequence of SEQ ID NO: 29, a nucleic acid sequence of SEQ ID NO: 20, and a nucleic acid sequence of SEQ ID NO: 26.

The recombinant microorganism according to the present disclosure has higher consumption efficiency of pentose than the wild-type microorganism has. The pentose is xylose, arabinose, ribose, ribose, etc., and, is preferably, xylose. The recombinant microorganism according to the present disclosure having the higher consumption efficiency of pentose than the wild-type microorganism has may mean that when culturing microorganisms in a mixed medium of glucose and pentose, the recombinant microorganism according to the present disclosure has higher pentose consumption than the wild-type microorganism has. In this connection, the pentose consumption refers to a percentage of pentose which is metabolized and is used for fermentation.

Further, the present disclosure relates to a method of obtaining a fermentation product, the method including culturing the recombinant microorganisms according to the present disclosure, thereby producing a culture; and obtaining a fermentation product from the culture.

The recombinant microorganism may be recombinant Escherichia coli or recombinant Clostridium. The culture step may be carried out in a conventional manner, and is not particularly limited. The fermentation product may be low-carbon alcohol having 6 or smaller carbons, an organic acid, and the like. For example, the fermentation product may be butanol, butanediol, propanol, propanediol, lactic acid, propionic acid, and the like.

[Modes]

Advantages and features according to the present disclosure, and methods for achieving them will be clarified with reference to embodiments described below in detail. However, the present disclosure is not limited to the embodiments disclosed below, but will be implemented in various different forms. The present embodiments are provided to allow the present disclosure to be complete, and to completely inform the skilled person to the art of the scope of the disclosure. The present disclosure is only defined by the scope of the claims.

<Materials and Methods>

As a mutant strain in which pta and buk were deleted, Clostridium acetobutylicum TM2-1-C(Accession No. KCTC 12604BP) was used.

In one example, in evaluating bio-butanol producing ability of recombinant C. acetobutylicum strain, alcohol selectivity (a percentage of a specific alcohol in a produced mixed solvent (ABE: acetone, butanol, ethanol)), butanol productivity and yield were calculated as follows.

Butanol selectivity (%):butanol production amount(g)/ABE production amount(g)×100

Ethanol selectivity (%):ethanol production amount(g)/ABE production amount(g)×100
Butanol productivity (g/L/h): amount of butanol produced per unit time and unit volume (in this connection, in batch and fed-batch culture methods, the butanol productivity is based on an exponential phase, and in a continuous culture method, the butanol productivity is calculated based on a cumulative ABE amount produced in an entire phase)

Yield (%):ABE production amount(g)/carbon source(g)×100
ABE productivity (g/L/h): amount of ABE produced per unit time and unit volume

### <Experimental Example 1> Preparation of pLK1-MCS Shuttle Plasmid

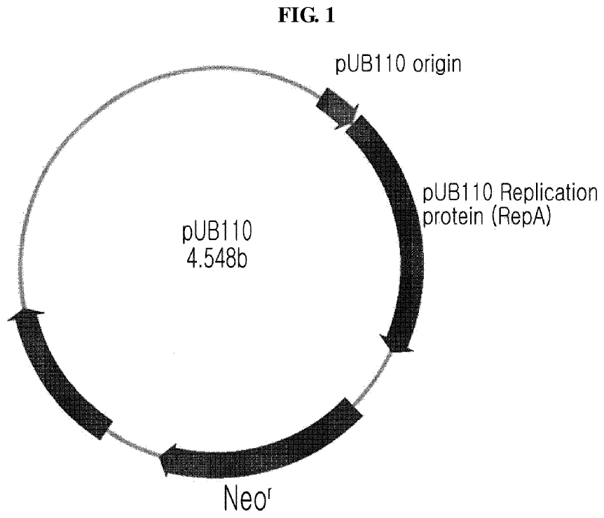

A pUB110 plasmid was Staphylococcus aureus-derived cryptic plasmid, and a gene map thereof is shown in FIG. 1. The shuttle plasmid was produced by amplifying a replication origin and a replication protein coding region of the pUB110 plasmid using a primer having a nucleic acid sequence of SEQ ID NO: 1 and a primer having a nucleic acid sequence of SEQ ID NO: 2, while using the pUB110 cryptic plasmid as a template. A nucleic acid sequence of a replication origin of the pUB110 plasmid is the same as SEQ ID NO: 3, and a nucleic acid sequence for encoding a replication protein (RepA) is the same as SEQ ID NO: 4, and an amino acid sequence of the replication protein is the same as SEQ ID NO: 5.

In this connection, 100 μl of a PCR reaction mixture was produced by adding dNTP 250 μM and the primers to 20 p mol, 1.5 mM MgCl₂, 10×buffer 10 μl, DNA template 100 ng, and 1 unit of pfu polymerase. After initial denaturation of the mixture at 95° C. for 5 minutes, denaturation at 95° C. for 1 minute, annealing at 58° C. for 1 minute, and polymerization at 72° C. for 2 minutes were repeated 30 times. PCR reaction in following Examples was performed in the same manner as the above method. The amplified DNA fragments were purified using a 1% agarose gel, and the DNA fragments were cleaved with SacI/BglII restriction enzyme.

TABLE 1

| | |
|---|---|
| SEQ ID NO 1 | ATAGAGCTCACGAAGTCGAGATCAGGGAATGAG |
| SEQ ID NO 2 | GCGAGATCTCTCGTCTTCCTAAGCATCCTTCAATCG |
| SEQ ID NO 3 | CTTGTTCTTTCTTATCTTGATACATATAGAAATAACGTCATTTTTATTTTAGTTGCTGAAAGGTGC |
| | GTTGAAGTGTTGGTATGTATGTGTTTTAAAGTATTGAAAACCCTTAAAATTGGTTGCACAGAAAA |
| | ACCCCATCTGTTAAAGTTATAAGTGACTAAACAAATAACTAAATAGA |
| SEQ ID NO 4 | ATGGGGGTTTCTTTTAATATTATGTGTCCTAATAGTAGCATTTATTCAGATGAAAAATCAAGGGT |
| | TTTAGTGGACAAGACAAAAAGTGGAAAAGTGAGACCATGGAGAGAAAAGAAAATCGCTAATG |
| | TTGATTACTTTGAACTTCTGCATATTCTTGAATTTAAAAAGGCTGAAAGAGTAAAAGATTGTGCT |
| | GAAATATTAGAGTATAAACAAAATCGTGAAACAGGCGAAAGAAAGTTGTATCGAGTGTGGTTT |
| | TGTAAATCCAGGCTTTGTCCAATGTGCAACTGGAGGAGAGCAATGAAACATGGCATTCAGTCA |
| | CAAAAGGTTGTTGCTGAAGTTATTAAACAAAAGCCAACAGTTCGTTGGTTGTTTCTCACATTAAC |
| | AGTTAAAAATGTTTATGATGGCGAAGAATTAAATAAGAGTTTGTCAGATATGGCTCAAGGATTT |
| | CGCCGAATGATGCAATATAAAAAAATTAATAAAAATCTTGTTGGTTTTATGCGTGCAACGGAAG |
| | TGACAATAAATAATAAAGATAATTCTTATAATCAGCACATGCATGTATTGGTATGTGTGGAACC |
| | AACTTATTTTAAGAATACAGAAAACTACGTGAATCAAAAACAATGGATTCAATTTTGGAAAAAG |
| | GCAATGAAATTAGACTATGATCCAAATGTAAAAGTTCAAATGATTCGACCGAAAAATAAATATA |
| | AATCGGATATACAATCGGCAATTGACGAAACTGCAAAATATCCTGTAAAGGATACGGATTTTAT |
| | GACCGATGATGAAGAAAAGAATTTGAAACGTTTGTCTGATTTGGAGGAAGGTTTACACCGTAA |
| | AAGGTTAATCTCCTATGGTGGTTTGTTAAAAGAAATACATAAAAAATTAAACCTTGATGACACA |
| | GAAGAAGGCGATTTGATTCATACAGATGATGACGAAAAAGCCGATGAAGATGGATTTTCTATTA |
| | TTGCAATGTGGAATTGGGAACGGAAAAATTATTTTATTAAAGAGTAG |
| SEQ ID NO 5 | MGVSFNIMCPNSSIYSDEKSRVLVDKTKSGKVRPWREKKIANVDYFELLHILEFKKAERVKDCAEILEY |
| | KQNRETGERKLYRVWFCKSRLCPMCNWRRAMKHGIQSQKVVAEVIKQKPTVRWLFLTLTVKNVYD |
| | GEELNKSLSDMAQGFRRMMQYKKINKNLVGFMRATEVTINNKDNSYNQHMHVLVCVEPTYFKNT |
| | ENYVNQKQWIQFWKKAMKLDYDPNVKVQMIRPKNKYKSDIQSAIDETAKYPVKDTDFMTDDEEK |
| | NLKRLSDLEEGLHRKRLISYGGLLKEIHKKLNLDDTEEGDLIHTDDDEKADEDGFSIIAMWNWERKNY |
| | FIKE |

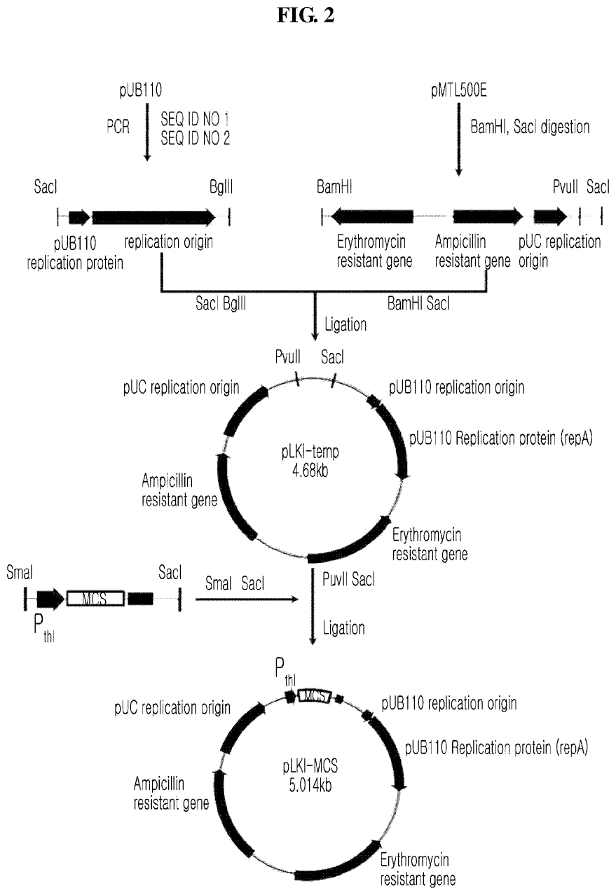

As shown in FIG. 2, pMTL500E plasmid was cleaved with SacI/BamHI restriction enzyme to obtain an ampicillin resistant gene, an erythromycin resistant gene, a replication origin site (pUC origin) of a pUC19 plasmid included in the pMTL500E plasmid, which in turn were purified using 1% agarose gel, and obtained. It was amplified using the PCR reaction, and ligated with DNA fragment (SEQ ID NO: 3 and 4) including a replication protein and a replication origin of the pUB110 plasmid and cleaved with SacI/BglII restriction enzyme, thereby preparing pLK1-temp. The prepared pLK1-temp was again cleaved with PvuII/SacI restriction enzyme and then was cloned with DNA fragments obtained by cleaving DNA (SEQ ID NO: 6) including thiolase promoter and multiple cloning site (MCS) chemically synthesized from the Bioneer company using SmaI/SacI. Thus, a final pLK1-MCS was prepared (SEQ ID NO: 10, FIG. 2). The pLK1-MCS includes a nucleic acid sequence (SEQ ID NO: 7) of the erythromycin resistant gene, a nucleic acid sequence (SEQ ID NO: 8) of the ampicillin resistant gene, a nucleic acid sequence (SEQ ID NO: 3) of a replication origin of pUB110 plasmid, a nucleic acid sequence (SEQ ID NO: 4) for encoding the replication protein (RepA), and a nucleic acid sequence (SEQ ID NO: 9) of the replication origin of pUC19 plasmid.

The pLK1-MCS (DNA plasmid) was deposited with the Korea Research Institute of Bioscience and Biotechnology on Feb. 4, 2015 (KCTC 12755BP).

TABLE 2

| | |
|---|---|
| SEQ ID NO 6 | ATACCCGGGCATGATTTTAAGGGGGTTAGCAGATGCATAAGTTTAATTTTTTTGTTAAAAAATAT |
| | TAAACTTTGTGTTTTTTTTAACAAAATATATTGATAAAAATAATAATAGTGGGTATAATTAAGTTG |
| | TTAGAGAAAACGTATAAATTAGGGATAAACTATGGAACTTATGAAATAGATTGAAATGGTTTAT |
| | CTGTTACCCCGTATCAAAATTTAGGAGGTTAGTTTAAACCTGCAGAGATCTCTCGAGGCGGCCG |
| | CGTCGACTCTAGACCCGGGAATTCACTGGCCGTCGTTTTACAACGTCGTGACTGGGAAAACCCT |
| | GGCGTTACCCAACTTAATCGCCTTGCAGCACATCCCCCTTTCGCCAGCTGGCGTAATAGCGAAG |
| | AGGCCCGCACCGATCGCCCTTCCCAACAGTTGCGCAGCCTGAATGGCGAATGGCGCCTGATGC |
| | GGTATTTTCTCCTTACGCATCTGTGCGGTATTTCACACCGAGCTCATA |
| SEQ ID NO 7 | ATGAACAAAAATATAAAATATTCTCAAAACTTTTTAACGAGTGAAAAAGTACTCAACCAAATAA |
| | TAAAACAATTGAATTTAAAAGAAACCGATACCGTTTACGAAATTGGAACAGGTAAAGGGCATTT |
| | AACGACGAAACTGGCTAAAATAAGTAAACAGGTAACGTCTATTGAATTAGACAGTCATCTATTC |
| | AACTTATCGTCAGAAAAATTAAAACTGAATACTCGTGTCACTTTAATTCACCAAGATATTCTACA |
| | GTTTCAATTCCCTAACAAACAGAGGTATAAAATTGTTGGGAGTATTCCTTACCATTTAAGCACAC |
| | AAATTATTAAAAAAGTGGTTTTTGAAAGCCATGCGTCTGACATCTATCTGATTGTTGAAGAAGG |
| | ATTCTACAAGCGTACCTTGGATATTCACCGAACACTAGGGTTGCTCTTGCACACTCAAGTCTCG |
| | ATTCAGCAATTGCTTAAGCTGCCAGCGGAATGCTTTCATCCTAAACCAAAAGTAAACAGTGTCT |
| | TAATAAAACTTACCCGCCATACCACAGATGTTCCAGATAAATATTGGAAGCTATATACGTACTTT |
| | GTTTCAAAATGGGTCAATCGAGAATATCGTCAACTGTTTACTAAAAATCAGTTTCATCAAGCAAT |
| | GAAACACGCCAAAGTAAACAATTTAAGTACCGTTACTTATGAGCAAGTATTGTCTATTTTTAATA |
| | GTTATCTATTATTTAACGGGAGGAAATAA |
| SEQ ID NO 8 | ATGAGTATTCAACATTTCCGTGTCGCCCTTATTCCCTTTTTTGCGGCATTTTGCCTTCCTGTTTTTG |
| | CTCACCCAGAAACGCTGGTGAAAGTAAAAGATGCTGAAGATCAGTTGGGTGCACGAGTGGGTT |
| | ACATCGAACTGGATCTCAACAGCGGTAAGATCCTTGAGAGTTTTCGCCCCGAAGAACGTTTTCC |
| | AATGATGAGCACTTTTAAAGTTCTGCTATGTGGCGCGGTATTATCCCGTATTGACGCCGGGCAA |
| | GAGCAACTCGGTCGCCGCATACACTATTCTCAGAATGACTTGGTTGAGTACTCACCAGTCACAG |
| | AAAAGCATCTTACGGATGGCATGACAGTAAGAGAATTATGCAGTGCTGCCATAACCATGAGTG |
| | ATAACACTGCGGCCAACTTACTTCTGACAACGATCGGAGGACCGAAGGAGCTAACCGCTTTTTT |
| | GCACAACATGGGGGATCATGTAACTCGCCTTGATCGTTGGGAACCGGAGCTGAATGAAGCCAT |
| | ACCAAACGACGAGCGTGACACCACGATGCCTGTAGCAATGGCAACAACGTTGCGCAAACTATT |
| | AACTGGCGAACTACTTACTCTAGCTTCCCGGCAACAATTAATAGACTGGATGGAGGCGGATAA |
| | AGTTGCAGGACCACTTCTGCGCTCGGCCCTTCCGGCTGGCTGGTTTATTGCTGATAAATCTGGA |
| | GCCGGTGAGCGTGGGTCTCGCGGTATCATTGCAGCACTGGGGCCAGATGGTAAGCCCTCCCGT |
| | ATCGTAGTTATCTACACGACGGGGAGTCAGGCAACTATGGATGAACGAAATAGACAGATCGCT |
| | GAGATAGGTGCCTCACTGATTAAGCATTGGTAA |
| SEQ ID NO | TCAAAGGATCTTCTTGAGATCCTTTTTTTTCTGCGCGTAATCTGCTGCTTGCAAACAAAAAAACCA |
| 9 | CCGCTACCAGCGGTGGTTTGTTTGCCGGATCAAGAGCTACCAACTCTTTTTCCGAAGGTAACTG |
| | GCTTCAGCAGAGCGCAGATACCAAATACTGTTCTTCTAGTGTAGCCGTAGTTAGGCCACCACTT |
| | CAAGAACTCTGTAGCACCGCCTACATACCTCGCTCTGCTAATCCTGTTACCAGTGGCTGCTGCC |
| | AGTGGCGATAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCTACACCGA |
| | ACTGAGATACCTACAGCGTGAGCTATGAGAAAGCGCCACGCTTCCCGAAGGGAGAAAGGCGG |
| | ACAGGTATCCGGTAAGCGGCAGGGTCGGAACAGGAGAGCGCACGAGGGAGCTTCCAGGGGG |
| | AAACGCCTGGTATCTTTATAGTCCTGTCGGGTTTCGCCACCTCTGACTTGAGCGTCGATTTTTGT |
| | GATGCTCGTCAGGGGGGCGGAGCCTATGGAAAAACGCCAGCAACG |
| SEQ ID NO | CATGATTTTAAGGGGGTTAGCAGATGCATAAGTTTAATTTTTTTGTTAAAAAATATTAAACTTTGT |
| 10 | GTTTTTTTTAACAAAATATATTGATAAAAATAATAATAGTGGGTATAATTAAGTTGTTAGAGAAA |
| | ACGTATAAATTAGGGATAAACTATGGAACTTATGAAATAGATTGAAATGGTTTATCTGTTACCCC |
| | GTATCAAAATTTAGGAGGTTAGTTTAAACCTGCAGAGATCTCTCGAGGCGGCCGCGTCGACTCT |
| | AGACCCGGGAATTCACTGGCCGTCGTTTTACAACGTCGTGACTGGGAAAACCCTGGCGTTACC |
| | CAACTTAATCGCCTTGCAGCACATCCCCCTTTCGCCAGCTGGCGTAATAGCGAAGAGGCCCGC |
| | ACCGATCGCCCTTCCCAACAGTTGCGCAGCCTGAATGGCGAATGGCGCCTGATGCGGTATTTTC |
| | TCCTTACGCATCTGTGCGGTATTTCACACCGAGCTCACGAAGTCGAGATCAGGGAATGAGTTTA |
| | TAAAATAAAAAAAGCACCTGAAAAGGTGTCTTTTTTTGATGGTTTTGAACTTGTTCTTTCTTATCT |
| | TGATACATATAGAAATAACGTCATTTTTATTTTAGTTGCTGAAAGGTGCGTTGAAGTGTTGGTAT |
| | GTATGTGTTTTAAAGTATTGAAAACCCTTAAAATTGGTTGCACAGAAAAACCCCATCTGTTAAAG |
| | TTATAAGTGACTAAACAAATAACTAAATAGATGGGGGTTTCTTTTAATATTATGTGTCCTAATAG |
| | TAGCATTTATTCAGATGAAAAATCAAGGGTTTTAGTGGACAAGACAAAAAGTGGAAAAGTGAG |
| | ACCATGGAGAGAAAAGAAAATCGCTAATGTTGATTACTTTGAACTTCTGCATATTCTTGAATTTA |
| | AAAAGGCTGAAAGAGTAAAAGATTGTGCTGAAATATTAGAGTATAAACAAAATCGTGAAACAG |
| | GCGAAAGAAAGTTGTATCGAGTGTGGTTTTGTAAATCCAGGCTTTGTCCAATGTGCAACTGGAG |
| | GAGAGCAATGAAACATGGCATTCAGTCACAAAAGGTTGTTGCTGAAGTTATTAAACAAAAGCC |
| | AACAGTTCGTTGGTTGTTTCTCACATTAACAGTTAAAAATGTTTATGATGGCGAAGAATTAAATA |
| | AGAGTTTGTCAGATATGGCTCAAGGATTTCGCCGAATGATGCAATATAAAAAAATTAATAAAAA |
| | TCTTGTTGGTTTTATGCGTGCAACGGAAGTGACAATAAATAATAAAGATAATTCTTATAATCAGC |
| | ACATGCATGTATTGGTATGTGTGGAACCAACTTATTTTAAGAATACAGAAAACTACGTGAATCA |
| | AAAACAATGGATTCAATTTTGGAAAAAGGCAATGAAATTAGACTATGATCCAAATGTAAAAGTT |
| | CAAATGATTCGACCGAAAAATAAATATAAATCGGATATACAATCGGCAATTGACGAAACTGCA |
| | AAATATCCTGTAAAGGATACGGATTTTATGACCGATGATGAAGAAAAGAATTTGAAACGTTTGT |
| | CTGATTTGGAGGAAGGTTTACACCGTAAAAGGTTAATCTCCTATGGTGGTTTGTTAAAAGAAAT |
| | ACATAAAAAATTAAACCTTGATGACACAGAAGAAGGCGATTTGATTCATACAGATGATGACGA |
| | AAAAGCCGATGAAGATGGATTTTCTATTATTGCAATGTGGAATTGGGAACGGAAAAATTATTTT |
| | ATTAAAGAGTAGTTCAACAAACGGGCCAGTTTGTTGAAGATTAGATGCTATAATTGTTATTAAA |
| | AGGATTGAAGGATGCTTAGGAAGACGAGAGATCCTAGCAGCACGCCATAGTGACTGGCGATG |
| | CTGTCGGAATGGACGATCAAATTCCCCGTAGGCGCTAGGGACCTCTTTAGCTCCTTGGAAGCTG |
| | TCAGTAGTATACCTAATAATTTATCTACATTCCCTTTAGTAACGTGTAACTTTCCAAATTTACAAA |
| | AGCGACTCATAGAATTATTTCCTCCCGTTAAATAATAGATAACTATTAAAAATAGACAATACTTG |
| | CTCATAAGTAACGGTACTTAAATTGTTTACTTTGGCGTGTTTCATTGCTTGATGAAACTGATTTTT |
| | AGTAAACAGTTGACGATATTCTCGATTGACCCATTTTGAAACAAAGTACGTATATAGCTTCCAAT |
| | ATTTATCTGGAACATCTGTGGTATGGCGGGTAAGTTTTATTAAGACACTGTTTACTTTTGGTTTAG |
| | GATGAAAGCATTCCGCTGGCAGCTTAAGCAATTGCTGAATCGAGACTTGAGTGTGCAAGAGCA |
| | ACCCTAGTGTTCGGTGAATATCCAAGGTACGCTTGTAGAATCCTTCTTCAACAATCAGATAGAT |
| | GTCAGACGCATGGCTTTCAAAAACCACTTTTTTAATAATTTGTGTGCTTAAATGGTAAGGAATAC |
| | TCCCAACAATTTTATACCTCTGTTTGTTAGGGAATTGAAACTGTAGAATATCTTGGTGAATTAAA |
| | GTGACACGAGTATTCAGTTTTAATTTTTCTGACGATAAGTTGAATAGATGACTGTCTAATTCAAT |
| | AGACGTTACCTGTTTACTTATTTTAGCCAGTTTCGTCGTTAAATGCCCTTTACCTGTTCCAATTTC |
| | GTAAACGGTATCGGTTTCTTTTAAATTCAATTGTTTTATTATTTGGTTGAGTACTTTTTCACTCGTT |
| | AAAAAGTTTTGAGAATATTTTATATTTTTGTTCATGTAATCACTCCTTCTTAATTACAAATTTTTAG |
| | CATCTAATTTAACTTCAATTCCTATTATACAAAATTTTAAGATACTGCACTATCAACACACTCTTA |
| | AGTTTGCTTCTAAGTCTTATTTCCATAACTTCTTTTACGTTTCCGCCATTCTTTGCTGTTTCGATTTT |
| | TATGATATGGTGCAAGTCAGCACGAACACGAACCGTCTTATCTCCCATTATATCTTTTTTTGCAC |
| | TGATTGGTGTATCATTTCGTTTTTCTTTTTATCCCGCAAGAGGCCCGGCAGTCAGGTGGCACTTTT |
| | CGGGGAAATGTGCGCGGAACCCCTATTTGTTTATTTTTCTAAATACATTCAAATATGTATCCGCT |
| | CATGAGACAATAACCCTGATAAATGCTTCAATAATATTGAAAAAGGAAGAGTATGAGTATTCAA |
| | CATTTCCGTGTCGCCCTTATTCCCTTTGCGGCATTTTGCCTTCCTGTTTTTGCTCACCCAGAAA |
| | CGCTGGTGAAAGTAAAAGATGCTGAAGATCAGTTGGGTGCACGAGTGGGTTACATCGAACTGG |
| | ATCTCAACAGCGGTAAGATCCTTGAGAGTTTTCGCCCCGAAGAACGTTTTCCAATGATGAGCAC |
| | TTTAAAGTTCTGCTATGTGGCGCGGTATTATCCCGTATTGACGCCGGGCAAGAGCAACTCGGT |
| | CGCCGCATACACTATTCTCAGAATGACTTGGTTGAGTACTCACCAGTCACAGAAAAGCATCTTA |
| | CGGATGGCATGACAGTAAGAGAATTATGCAGTGCTGCCATAACCATGAGTGATAACACTGCGG |
| | CCAACTTACTTCTGACAACGATCGGAGGACCGAAGGAGCTAACCGCTTTTTTGCACAACATGG |
| | GGGATCATGTAACTCGCCTTGATCGTTGGGAACCGGAGCTGAATGAAGCCATACCAAACGACG |
| | AGCGTGACACCACGATGCCTGTAGCAATGGCAACAACGTTGCGCAAACTATTAACTGGCGAAC |
| | TACTTACTCTAGCTTCCCGGCAACAATTAATAGACTGGATGGAGGCGGATAAAGTTGCAGGAC |
| | CACTTCTGCGCTCGGCCCTTCCGGCTGGCTGGTTTATTGCTGATAAATCTGGAGCCGGTGAGCG |
| | TGGGTCTCGCGGTATCATTGCAGCACTGGGGCCAGATGGTAAGCCCTCCCGTATCGTAGTTATC |
| | TACACGACGGGGAGTCAGGCAACTATGGATGAACGAAATAGACAGATCGCTGAGATAGGTGC |
| | CTCACTGATTAAGCATTGGTAACTGTCAGACCAAGTTTACTCATATATACTTTAGATTGATTTAA |
| | AACTTCATTTTTAATTTAAAAGGATCTAGGTGAAGATCCTTTTTGATAATCTCATGACCAAAATCC |
| | CTTAACGTGAGTTTTCGTTCCACTGAGCGTCAGACCCCGTAGAAAAGATCAAAGGATCTTCTTG |
| | AGATCCTTTTTTTCTGCGCGTAATCTGCTGCTTGCAAACAAAAAAACCACCGCTACCAGCGGTG |
| | GTTTGTTTGCCGGATCAAGAGCTACCAACTCTTTTTCCGAAGGTAACTGGCTTCAGCAGAGCGC |
| | AGATACCAAATACTGTTCTTCTAGTGTAGCCGTAGTTAGGCCACCACTTCAAGAACTCTGTAGC |
| | ACCGCCTACATACCTCGCTCTGCTAATCCTGTTACCAGTGGCTGCTGCCAGTGGCGATAAGTCG |
| | TGTCTTACCGGGTTGGACTCAAGACGATAGTTACCGGATAAGGCGCAGCGGTCGGGCTGAACG |
| | GGGGGTTCGTGCACACAGCCCAGCTTGGAGCGAACGACCTACACCGAACTGAGATACCTACA |
| | GCGTGAGCTATGAGAAAGCGCCACGCTTCCCGAAGGGAGAAAGGCGGACAGGTATCCGGTAA |
| | GCGGCAGGGTCGGAACAGGAGAGCGCACGAGGGAGCTTCCAGGGGGAAACGCCTGGTATCT |
| | TTATAGTCCTGTCGGGTTTCGCCACCTCTGACTTGAGCGTCGATTTTTGTGATGCTCGTCAGGGG |
| | GGCGGAGCCTATGGAAAAACGCCAGCAACGCGGCCTTTTTACGGTTCCTGGCCTTTTGCTGGC |
| | CTTTTGCTCACATGTTCTTTCCTGCGTTATCCCCTGATTCTGTGGATAACCGTATTACCGCCTTTG |
| | AGTGAGCTGATACCGCTCGCCGCAGCCGAACGACCGAGCGCAGCGAGTCAGTGAGCGAGGA |
| | AGCGGAAGAGCGCCCAATACGCAAACCGCCTCTCCCCGCGCGTTGGCCGATTCATTAATGCAG |
| | GGG |

### <Experimental Example 2> Preparation of pCK2-MCS Shuttle Plasmid

<2-1> Amplification of Replication Origin of pLK1-MCS Plasmid

The replication origin site of the pUC19 plasmid, the thiloase promoter region, the MCS site, the replication origin of the pUB110 plasmid, and the replication protein coding region of the pLK1-MCS plasmid prepared in <Experimental Example 1> were amplified and thus obtained. A nucleic acid sequence of the thus obtained DNA fragment is the same as SEQ ID NO: 14.

In this connection, the amplification was conducted using a primer having a nucleic acid sequence of SEQ ID NO: 11 and a primer having a nucleic acid sequence of SEQ ID NO: 12 while using the pLK1-MCS plasmid as a template. The nucleic acid sequence of the replication origin site of the pUC19 plasmid is the same as SEQ ID NO: 9. The nucleic acid sequence of the replication origin of pUB110 plasmid is the same as SEQ ID NO: 3. The nucleic acid sequence of the coding region of the replication protein is the same as SEQ ID NO: 4. The amino acid sequence of the replication protein is the same as SEQ ID NO: 5. The nucleic acid sequence of the thiolase promoter region is the same as SEQ ID NO: 13. The nucleic acid sequence of the thiolase promoter region and multiple cloning site (MCS) is the same as SEQ ID NO: 6.

In this connection, 50 μl of a PCR reaction mixture was produced by adding dNTP 100 μM and the primers to 10 p mol, 10×buffer 5 ul, DNA template 100 ng, and 0.5 unit of pfu polymerase. After initial denaturation of the mixture at 95° C. for 5 minutes, denaturation at 95° C. for 1 minute, annealing at 58° C. for 1 minute, and polymerization at 72° C. for 3 minutes were repeated 30 times. The amplified DNA fragments were purified using a 1% agarose gel, and the DNA fragments were cleaved with StuI/BamHI restriction enzyme.

TABLE 3

| | |
|---|---|
| SEQ ID NO 11 | CTCAGGCCTGTGAGTTTTCGTTCCACTGAGCGTCAGACC |
| SEQ ID NO 12 | GCGGGATCCCTCGTCTTCCTAAGCATCCTTCAATCC |
| SEQ ID NO 13 | CATGATTTTAAGGGGGTTAGCAGATGCATAAGTTTAATTTTTTTGTTAAAAAATATTAAACTTT |
| | GTGTTTTTTTTAACAAAATATATTGATAAAAATAATAATAGTGGGTATAATTAAGTTGTTAGA |
| | GAAAACGTATAAATTAGGGATAAACTATGGAACTTATGAAATAGATTGAAATGGTTTATCTG |
| | TTACCCCGTATCAAAATTT |
| SEQ ID NO 14 | AGGCCTATGGGGGTTTCTTTTAATATTATGTGTCCTAATAGTAGCATTTATTCAGATGAAAAA |
| | TCAAGGGTTTTAGTGGACAAGACAAAAAGTGGAAAAGTGAGACCATGGAGAGAAAAGAAA |
| | ATCGCTAATGTTGATTACTTTGAACTTCTGCATATTCTTGAATTTAAAAAGGCTGAAAGAGTA |
| | AAAGATTGTGCTGAAATATTAGAGTATAAACAAAATCGTGAAACAGGCGAAAGAAAGTTGT |
| | ATCGAGTGTGGTTTTGTAAATCCAGGCTTTGTCCAATGTGCAACTGGAGGAGAGCAATGAA |
| | ACATGGCATTCAGTCACAAAAGGTTGTTGCTGAAGTTATTAAACAAAAGCCAACAGTTCGTT |
| | GGTTGTTTCTCACATTAACAGTTAAAAATGTTTATGATGGCGAAGAATTAAATAAGAGTTTGT |
| | CAGATATGGCTCAAGGATTTCGCCGAATGATGCAATATAAAAAAATTAATAAAAATCTTGTT |
| | GGTTTTATGCGTGCAACGGAAGTGACAATAAATAATAAAGATAATTCTTATAATCAGCACAT |
| | GCATGTATTGGTATGTGTGGAACCAACTTATTTTAAGAATACAGAAAACTACGTGAATCAAA |
| | AACAATGGATTCAATTTTGGAAAAAGGCAATGAAATTAGACTATGATCCAAATGTAAAAGTT |
| | CAAATGATTCGACCGAAAAATAAATATAAATCGGATATACAATCGGCAATTGACGAAACTG |
| | CAAAATATCCTGTAAAGGATACGGATTTTATGACCGATGATGAAGAAAAGAATTTGAAACG |
| | TTTGTCTGATTTGGAGGAAGGTTTACACCGTAAAAGGTTAATCTCCTATGGTGGTTTGTTAAA |
| | AGAAATACATAAAAAATTAAACCTTGATGACACAGAAGAAGGCGATTTGATTCATACAGAT |
| | GATGACGAAAAAGCCGATGAAGATGGATTTTCTATTATTGCAATGTGGAATTGGGAACGGA |
| | AAAATTATTTTATTAAAGAGTAGTTCAACAAACGGGCCAGTTTGTTGAAGATTAGATGCTAT |
| | AATTGTTATTAAAAGGATTGAAGGATGCTTAGGAAGACGAGAGATCCTAGCAGCACGCCAT |
| | AGTGACTGGCGATGCTGTCGGAATGGACGATCAAATTCCCCGTAGGCGCTAGGGACCTCTT |
| | TAGCTCCTTGGAAGCTGTCAGTAGTATACCTAATAATTTATCTACATTCCCTTTAGTAACGTG |
| | TAACTTTCCAAATTTACAAAAGCGACTCATAGAATTATTTCCTCCCGTTAAATAATAGATAAC |
| | TATTAAAAATAGACAATACTTGCTCATAAGTAACGGTACTTAAATTGTTTACTTTGGCGTGTT |
| | TCATTGCTTGATGAAACTGATTTTTAGTAAACAGTTGACGATATTCTCGATTGACCCATTTTG |
| | AAACAAAGTACGTATATAGCTTCCAATATTTATCTGGAACATCTGTGGTATGGCGGGTAAGT |
| | TTTATTAAGACACTGTTTACTTTTGGTTTAGGATGAAAGCATTCCGCTGGCAGCTTAAGCAAT |
| | TGCTGAATCGAGACTTGAGTGTGCAAGAGCAACCCTAGTGTTCGGTGAATATCCAAGGTAC |
| | GCTTGTAGAATCCTTCTTCAACAATCAGATAGATGTCAGACGCATGGCTTTCAAAAACCACT |
| | TTTTTAATAATTTGTGTGCTTAAATGGTAAGGAATACTCCCAACAATTTTATACCTCTGTTTGT |
| | TAGGGAATTGAAACTGTAGAATATCTTGGTGAATTAAAGTGACACGAGTATTCAGTTTTAAT |
| | TTTTCTGACGATAAGTTGAATAGATGACTGTCTAATTCAATAGACGTTACCTGTTTACTTATTT |
| | TAGCCAGTTTCGTCGTTAAATGCCCTTTACCTGTTCCAATTTCGTAAACGGTATCGGTTTCTTT |
| | TAAATTCAATTGTTTTATTATTTGGTTGAGTACTTTTTCACTCGTTAAAAAGTTTTGAGAATATT |
| | TTATATTTTTGTTCATGTAATCACTCCTTCTTAATTACAAATTTTTAGCATCTAATTTAACTTCA |
| | ATTCCTATTATACAAAATTTTAAGATACTGCACTATCAACACACTCTTAAGTTTGCTTCTAAGT |
| | CTTATTTCCATAACTTCTTTTACGTTTCCGCCATTCTTTGCTGTTTCGATTTTTATGATATGGTG |
| | CAAGTCAGCACGAACACGAACCGTCTTATCTCCCATTATATCTTTTTTTGCACTGATTGGTGT |
| | ATCATTTCGTTTTTCTTTTTATCCCGCAAGAGGCCCGGCAGTCAGGTGGCACTTTTCGGGGA |
| | AATGTGCGCGGAACCCCTATTTGTTTATTTTTCTAAATACATTCAAATATGTATCCGCTCATG |
| | AGACAATAACCCTGATAAATGCTTCAATAATATTGAAAAAGGAAGAGTATGAGTATTCAAC |
| | ATTTCCGTGTCGCCCTTATTCCCTTTTTTGCGGCATTTTGCCTTCCTGTTTTTGCTCACCCAGA |
| | AACGCTGGTGAAAGTAAAAGATGCTGAAGATCAGTTGGGTGCACGAGTGGGTTACATCGA |
| | ACTGGATCTCAACAGCGGTAAGATCCTTGAGAGTTTTCGCCCCGAAGAACGTTTTCCAATGA |
| | TGAGCACTTTTAAAGTTCTGCTATGTGGCGCGGTATTATCCCGTATTGACGCCGGGCAAGAG |
| | CAACTCGGTCGCCGCATACACTATTCTCAGAATGACTTGGTTGAGTACTCACCAGTCACAGA |
| | AAAGCATCTTACGGATGGCATGACAGTAAGAGAATTATGCAGTGCTGCCATAACCATGAGT |
| | GATAACACTGCGGCCAACTTACTTCTGACAACGATCGGAGGACCGAAGGAGCTAACCGCTT |
| | TTTTGCACAACATGGGGGATCATGTAACTCGCCTTGATCGTTGGGAACCGGAGCTGAATGA |
| | AGCCATACCAAACGACGAGCGTGACACCACGATGCCTGTAGCAATGGCAACAACGTTGCG |
| | CAAACTATTAACTGGCGAACTACTTACTCTAGCTTCCCGGCAACAATTAATAGACTGGATGG |
| | AGGCGGATAAAGTTGCAGGACCACTTCTGCGCTCGGCCCTTCCGGCTGGCTGGTTTATTGCT |
| | GATAAATCTGGAGCCGGTGAGCGTGGGTCTCGCGGTATCATTGCAGCACTGGGGCCAGAT |
| | GGTAAGCCCTCCCGTATCGTAGTTATCTACACGACGGGGAGTCAGGCAACTATGGATGAAG |
| | GAAATAGACAGATCGCTGAGATAGGTGCCTCACTGATTAAGCATTGGTAACTGTCAGACCA |
| | AGTTTACTCATATATACTTTAGATTGATTTAAAACTTCATTTTTAATTTAAAAGGATCTAGGTG |
| | AAGATCCTTTTTGATAATCTCATGACCAAAATCCCTTAACGTGAGTTTTCGTTCCACTGAGCG |
| | TCAGACCCCGTAGAAAAGATCAAAGGATCTTCTTGAGATCCTTTTTTTCTGCGCGTAATCTGC |
| | TGCTTGCAAACAAAAAAACCACCGCTACCAGCGGTGGTTTGTTTGCCGGATCAAGAGCTAC |
| | CAACTCTTTTTCCGAAGGTAACTGGCTTCAGCAGAGCGCAGATACCAAATACTGTTCTTCTA |
| | GTGTAGCCGTAGTTAGGCCACCACTTCAAGAACTCTGTAGCACCGCCTACATACCTCGCTCT |
| | GCTAATCCTGTTACCAGTGGCTGCTGCCAGTGGCGATAAGTCGTGTCTTACCGGGTTGGACT |
| | CAAGACGATAGTTACCGGATAAGGCGCAGCGGTCGGGCTGAACGGGGGGTTCGTGCACAC |
| | AGCCCAGCTTGGAGCGAACGACCTACACCGAACTGAGATACCTACAGCGTGAGCTATGAG |
| | AAAGCGCCACGCTTCCCGAAGGGAGAAAGGCGGACAGGTATCCGGTAAGCGGCAGGGTC |
| | GGAACAGGAGAGCGCACGAGGGAGCTTCCAGGGGGAAACGCCTGGTATCTTTATAGTCCT |
| | GTCGGGTTTCGCCACCTCTGACTTGAGCGTCGATTTTTGTGATGCTCGTCAGGGGGGCGGA |
| | GCCTATGGAAAAACGCCAGCAACGGGATCC |

<2-2> Chloramphenicol Resistant Gene Amplification

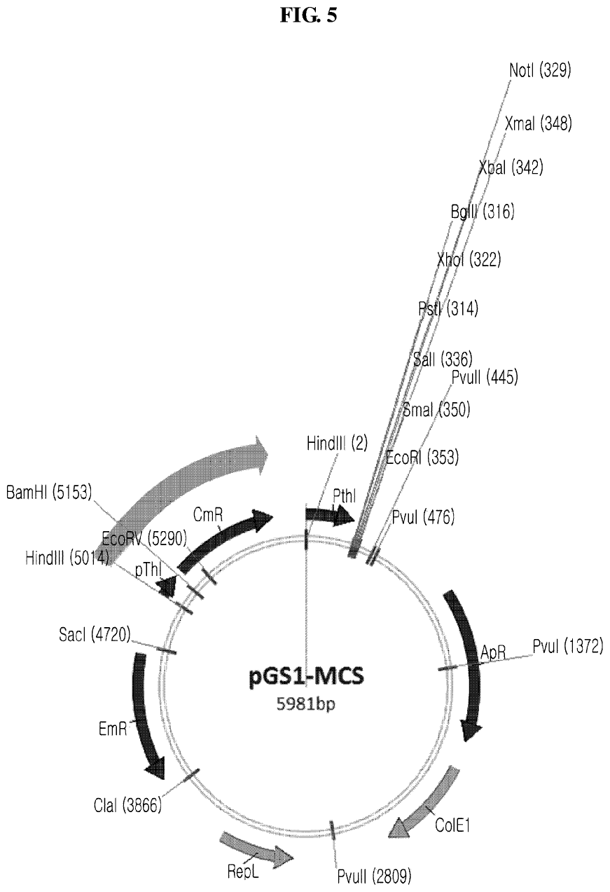

A chloramphenicol resistant gene included in pGS1-MCS plasmid was amplified and thus obtained. In this connection, the amplifying was conducted via PCR reaction using primers having nucleic acid sequences of SEQ ID NO: 15 and SEQ ID NO: 16. The amplified chloramphenicol resistant gene was cleaved using BglII/Xmal restriction enzyme, thereby to obtain a DNA fragment. A sequence of the amplified DNA fragment is the same as SEQ ID NO: 17 (Table 4). The pGS1-MCS plasmid includes a replication origin of a pIM13 plasmid and a nucleic acid sequence for encoding a replication protein (J. Microbiol. Biotechnol. (2015), 25 (10), 1702-1708, J. Microbiol. Biotechnol. (2016), 26 (4), 725-729, etc.). A structure thereof is shown in FIG. 5. However, in accordance with the present disclosure, to obtain a nucleic acid sequence of the chloramphenicol resistant gene, the pGS1-MCS plasmid was used. However, when the nucleic acid sequence of SEQ ID NO: 17 is obtained, it is not necessary to use the pGS1-MCS plasmid. Rather, other suitable plasmids may be used. Alternatively, the sequence may be synthesized and then used.

TABLE 4

| | |
|---|---|
| SEQ ID NO | ATAAGATCTCGACTTTTTAACAAAATATATTG |
| 15 | |
| SEQ ID NO | ATACCCGGGCAGCTCTAGGCAATATTATATCTGCAA |
| 16 | |
| SEQ ID NO | AGATCTCGACTTTTTAACAAAATATATTGATAAAAATAATAATAGTGGGTATAATTAAGTTGTTAG |
| 17 | AGAAAACGTATAAATTAGGGATAAACTATGGAACTTATGAAATAGATTGAAATGGTTTATCTGTT |
| | ACCCCGTAGGATCCACTTGAATTTAAAAGGAGGGAACTTAGATGGTATTTGAAAAAATTGATAAA |
| | AATAGTTGGAACAGAAAAGAGTATTTTGACCACTACTTTGCAAGTGTACCTTGTACATACAGCAT |
| | GACCGTTAAAGTGGATATCACACAAATAAAGGAAAAGGGAATGAAACTATATCCTGCAATGCTTT |
| | ATTATATTGCAATGATTGTAAACCGCCATTCAGAGTTTAGGACGGCAATCAATCAAGATGGTGAA |
| | TTGGGGATATATGATGAGATGATACCAAGCTATACAATATTTCACAATGATACTGAAACATTTTCC |
| | AGCCTTTGGACTGAGTGTAAGTCTGACTTTAAATCATTTTTAGCAGATTATGAAAGTGATACGCAA |
| | CGGTATGGAAACAATCATAGAATGGAAGGAAAGCCAAATGCTCCGGAAAACATTTTAATGTAT |
| | CTATGATACCGTGGTCAACCTTCGATGGCTTTAATCTGAATTTGCAGAAAGGATATGATTATTTGA |
| | TTCCTATTTTTACTATGGGGAAATATTATAAAGAAGATAACAGAATTATACTCCCTTTGGCAATTCA |
| | AGTTCATCACGCAGTATGTGACGGATTTCACATTTGCCGTTTTGTAAACGAATTGCAGGAATTGAT |
| | AAATAGTTAACTTCAGGTTTGTCTGTAACTGGCGCCACTTAATGATTTGCCAGTAAAAGAGATTGT |
| | TTCTAGCTCTCACATTCTTGCAGATATAATATTGCCTAGAGCTGCCCGGG |

<2-3> Ligation Between pLK1-MCS Plasmid and Chloramphenicol Resistant Gene Sequences

The DNA fragment amplified in the above <2-1> and the chloramphenicol gene portion amplified in the above <2-2> were ligated with each other using a T4 ligase enzyme, thereby producing a ligation product of a pCK2-temp.

<2-4> Insertion of Terminator of Adc Gene

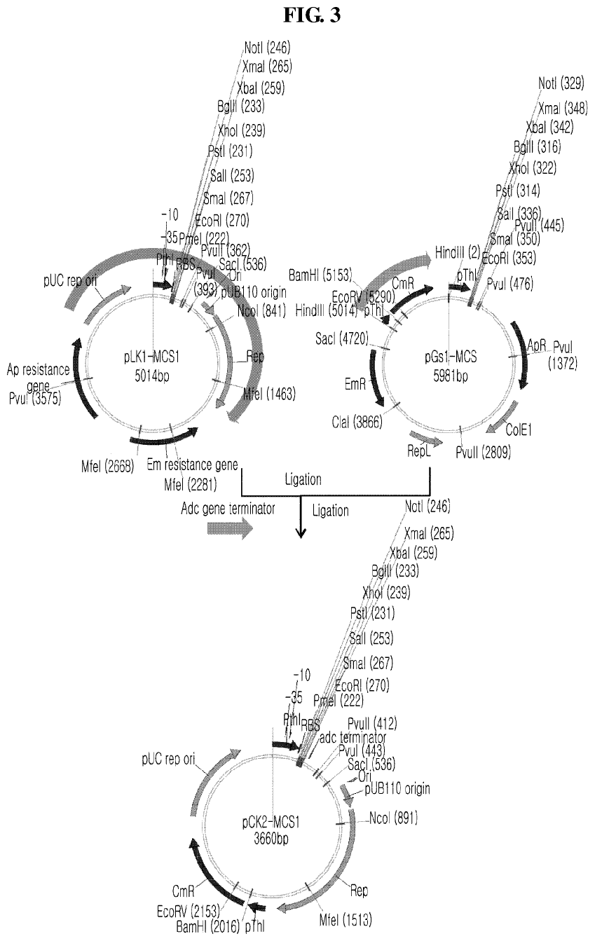

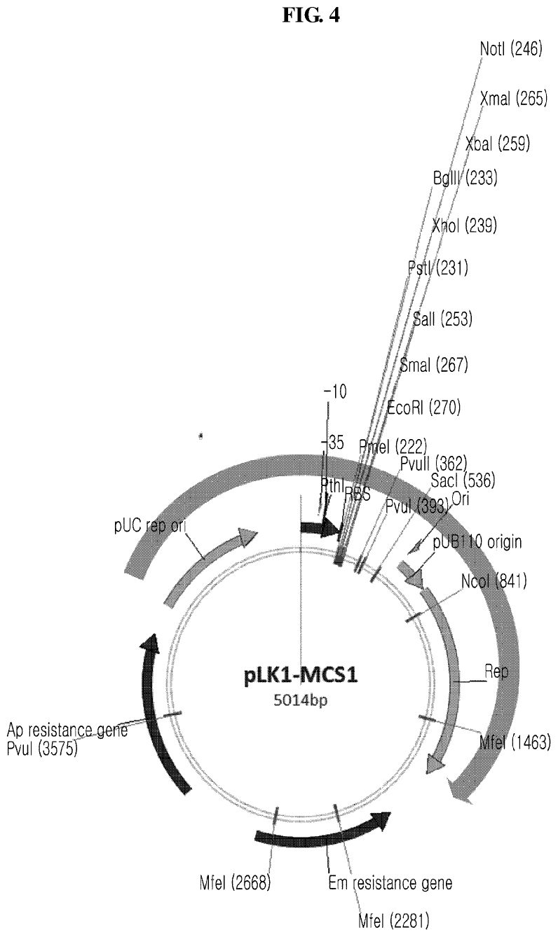

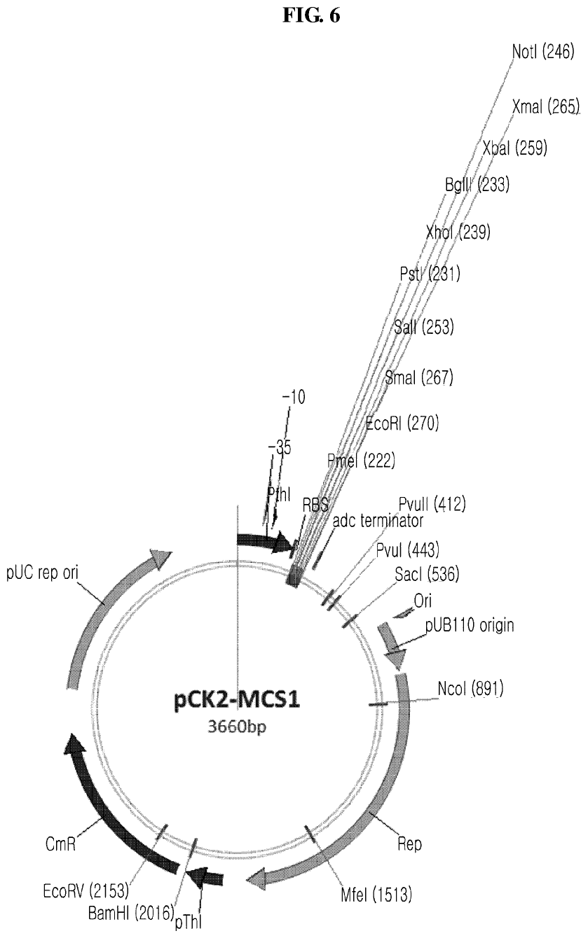

A DNA sequence (SEQ ID NO: 18) including a transcription terminator (Adc terminator) of the acetoacetate decarboxylase (Adc) gene of the Clostridium acetobutylicum chemically synthesized from the Bioneer company was obtained. The DNA sequence (SEQ ID NO: 18) was cleaved with EcoRI/PvuII restriction enzyme to obtain a first DNA fragment. On the other hand, the pCK2-temp ligation product prepared in <2-3> was cleaved with EcoRI/PvuII restriction enzyme to produce a second DNA fragment. Then, the first and second DNA fragments were cloned with each other. Finally, a pCK2-MCS shuttle plasmid was prepared (SEQ ID NO: 19, Table 5, FIG. 3 to FIG. 6). When the terminator of the Adc gene is inserted, and when the shuttle plasmid is introduced into microorganisms, expression of the introduced gene into the microorganisms occurs normally via the shuttle plasmid in the microorganisms. Thus, a protein is produced properly. The produced pCK2-MCS shuttle plasmid was deposited with the Korea Research Institute of Bioscience and Biotechnology on May 16, 2017 (Accession number: KCTC13267BP).

TABLE 5

| | |
|---|---|
| SEQ ID NO 18 | GAATTCTAAAAATAAGAGTTACCTTAAATGGTAACTCTTATTTTTTTAATATTGTTACTGG |
| | CCGTCGTTTTACAACGTCGTGACTGGGAAAACCCTGGCGTTACCCAACTTAATCGCCTTG |
| | CAGCACATCCCCCTTTCGCCAGCTG |
| SEQ ID NO 19 | CATGATTTTAAGGGGGTTAGCAGATGCATAAGTTTAATTTTTTTGTTAAAAAATATTAAAC |
| | TTTGTGTTTTTTTTAACAAAATATATTGATAAAAATAATAATAGTGGGTATAATTAAGTTGT |
| | TAGAGAAAACGTATAAATTAGGGATAAACTATGGAACTTATGAAATAGATTGAAATGGT |
| | TTATCTGTTACCCCGTATCAAAATTTAGGAGGTTAGTTTAAACCTGCAGAGATCTCTCGA |
| | GGCGGCCGCGTCGACTCTAGACCCGGGAATTCTAAAAATAAGAGTTACCTTAAATGGTA |
| | ACTCTTATTTTTTTAATATTGTTACTGGCCGTCGTTTTACAACGTCGTGACTGGGAAAACC |
| | CTGGCGTTACCCAACTTAATCGCCTTGCAGCACATCCCCCTTTCGCCAGCTGGCGTAATA |
| | GCGAAGAGGCCCGCACCGATCGCCCTTCCCAACAGTTGCGCAGCCTGAATGGCGAATG |
| | GCGCCTGATGCGGTATTTTCTCCTTACGCATCTGTGCGGTATTTCACACCGAGCTCACGA |
| | AGTCGAGATCAGGGAATGAGTTTATAAAATAAAAAAAGCACCTGAAAAGGTGTCTTTTT |
| | TTGATGGTTTTGAACTTGTTCTTTCTTATCTTGATACATATAGAAATAACGTCATTTTTATTT |
| | TAGTTGCTGAAAGGTGCGTTGAAGTGTTGGTATGTATGTGTTTTAAAGTATTGAAAACCC |
| | TTAAAATTGGTTGCACAGAAAAACCCCATCTGTTAAAGTTATAAGTGACTAAACAAATAA |
| | CTAAATAGATGGGGGTTTCTTTTAATATTATGTGTCCTAATAGTAGCATTTATTCAGATGA |
| | AAAATCAAGGGTTTTAGTGGACAAGACAAAAAGTGGAAAAGTGAGACCATGGAGAGAA |
| | AAGAAAATCGCTAATGTTGATTACTTTGAACTTCTGCATATTCTTGAATTTAAAAAGGCTG |
| | AAAGAGTAAAAGATTGTGCTGAAATATTAGAGTATAAACAAAATCGTGAAACAGGCGA |
| | AAGAAAGTTGTATCGAGTGTGGTTTTGTAAATCCAGGCTTTGTCCAATGTGCAACTGGAG |
| | GAGAGCAATGAAACATGGCATTCAGTCACAAAAGGTTGTTGCTGAAGTTATTAAACAAA |
| | AGCCAACAGTTCGTTGGTTGTTTCTCACATTAACAGTTAAAAATGTTTATGATGGCGAAG |
| | AATTAAATAAGAGTTTGTCAGATATGGCTCAAGGATTTCGCCGAATGATGCAATATAAAA |
| | AAATTAATAAAAATCTTGTTGGTTTTATGCGTGCAACGGAAGTGACAATAAATAATAAAG |
| | ATAATTCTTATAATCAGCACATGCATGTATTGGTATGTGTGGAACCAACTTATTTTAAGAA |
| | TACAGAAAACTACGTGAATCAAAAACAATGGATTCAATTTTGGAAAAAGGCAATGAAAT |
| | TAGACTATGATCCAAATGTAAAAGTTCAAATGATTCGACCGAAAAATAAATATAAATCG |
| | GATATACAATCGGCAATTGACGAAACTGCAAAATATCCTGTAAAGGATACGGATTTTAT |
| | GACCGATGATGAAGAAAAGAATTTGAAACGTTTGTCTGATTTGGAGGAAGGTTTACACC |
| | GTAAAAGGTTAATCTCCTATGGTGGTTTGTTAAAAGAAATACATAAAAAATTAAACCTTG |
| | ATGACACAGAAGAAGGCGATTTGATTCATACAGATGATGACGAAAAAGCCGATGAAGA |
| | TGGATTTTCTATTATTGCAATGTGGAATTGGGAACGGAAAAATTATTTTATTAAAGAGTA |
| | GTTCAACAAACGGGCCAGTTTGTTGAAGATTAGATGCTATAATTGTTATTAAAAGGATTG |
| | AAGGATGCTTAGGAAGACGAGGGATCTCGACnTTTAACAAAATATATTGATAAAAATA |
| | ATAATAGTGGGTATAATTAAGTTGTTAGAGAAAACGTATAAATTAGGGATAAACTATGG |
| | AACrTATGAAATAGATTGAAATGGTTTATCTGTTACCCCGTAGGATCCACTTGAATTTAA |
| | AAGGAGGGAACTTAGATGGTATTTGAAAAAATTGATAAAAATAGTTGGAACAGAAAAG |
| | AGTATTTTGACCACTACTTTGCAAGTGTACCTTGTACATACAGCATGACCGTTAAAGTGG |
| | ATATCACACAAATAAAGGAAAAGGGAATGAAACTATATCCTGCAATGCTTTATTATATTG |
| | CAATGATTGTAAACCGCCATTCAGAGTTTAGGACGGCAATCAATCAAGATGGTGAATTG |
| | GGGATATATGATGAGATGATACCAAGCTATACAATATTTCACAATGATACTGAAACATTT |
| | TCCAGCCTTTGGACTGAGTGTAAGTCTGACTTTAAATCATTTTTAGCAGATTATGAAAGT |
| | GATACGCAACGGTATGGAAACAATCATAGAATGGAAGGAAAGCCAAATGCTCCGGAAA |
| | ACATTTTTAATGTATCTATGATACCGTGGTCAACCTTCGATGGCTTTAATCTGAATTTGCA |
| | GAAAGGATATGAnATTTGATTCCTATTTTTACTATGGGGAAATATTATAAAGAAGATAA |
| | CAGAATTATACTCCCTTTGGCAATTCAAGTTCATCACGCAGTATGTGACGGATTTCACAT |
| | TTGCCGTTTTGTAAACGAATTGCAGGAATTGATAAATAGTTAACTTCAGGTTTGTCTGTAA |
| | CTGGCGCCACTTAATGATTTGCCAGTAAAAGAGATTGTTTCTAGCTCTCACATTCTTGCA |
| | GATATAATAnGCCTAGAGCTGCCCCCTGTGAGTTTTCGTTCCACTGAGCGTCAGACCCC |
| | GTAGAAAAGATCAAAGGATCTTCTTGAGATCCTTTTTTTCTGCGCGTAATCTGCTGCTTGC |
| | AAACAAAAAAACCACCGCTACCAGCGGTGGTTTGTTTGCCGGATCAAGAGCTACCAACT |
| | CTTTTTCCGAAGGTAACTGGCTTCAGCAGAGCGCAGATACCAAATACTGTTCTTCTAGTG |
| | TAGCCGTAGTTAGGCCACCACTTCAAGAACTCTGTAGCACCGCCTACATACCTCGCTCTG |
| | CTAATCCTGTTACCAGTGGCTGCTGCCAGTGGCGATAAGTCGTGTCTTACCGGGTTGGA |
| | CTCAAGACGATAGTTACCGGATAAGGCGCAGCGGTCGGGCTGAACGGGGGGTTCGTGC |
| | ACACAGCCCAGCTTGGAGCGAACGACCTACACCGAACTGAGATACCTACAGCGTGAGC |
| | TATGAGAAAGCGCCACGCTTCCCGAAGGGAGAAAGGCGGACAGGTATCCGGTAAGCG |
| | GCAGGGTCGGAACAGGAGAGCGCACGAGGGAGCTTCCAGGGGGAAACGCCTGGTATC |
| | TTTATAGTCCTGTCGGGTTTCGCCACCTCTGACTTGAGCGTCGATTTTTGTGATGCTCGTC |
| | AGGGGGGCGGAGCCTATGGAAAAACGCCAGCAACGCGGCCTTTTTACGGTTCCTGGCC |
| | TTTTGCTGGCCTTTTGCTCACATGTTCTTTCCTGCGTTATCCCCTGATTCTGTGGATAACCG |
| | TATTACCGCCTTTGAGTGAGCTGATACCGCTCGCCGCAGCCGAACGACCGAGCGCAGC |
| | GAGTCAGTGAGCGAGGAAGCGGAAGAGCGCCCAATACGCAAACCGCCTCTCCCCGCG |
| | CGTTGGCCGATTCATTAATGCAGGGG |

### <Experimental Example 3> Evaluation of pCK2-MCS Shuttle Plasmid

Characteristics of the pCK2-MCS shuttle plasmid produced in the above <Experimental Example 2> were evaluated. In this connection, a pLK1-MCS shuttle plasmid was used as a control.

<3-1> Evaluation of Plasmid Size

A size of the pCK2-MCS shuttle plasmid and a size of the pLK1-MCS shuttle plasmid were compared with each other.

The size of the pLK1-MCS shuttle plasmid was 5014 bp. The size of the pCK2-MCS shuttle plasmid was 3660 bp. Therefore, the pCK2-MCS shuttle plasmid was identified as having a fairly small size.

<3-2> Production of Transformant

Production of transformant of pCK2-MCS shuttle plasmid

First, the pCK2-MCS shuttle plasmid was introduced into the Clostridium acetobutylicum ATCC 824 strain to prepare a transformed recombinant microorganism. A specific method is as follows. The Clostridium acetobutylicum microorganism was cultured in 100 ml of CGM (Clostridium Growth Media) liquid medium (0.75 g/L K₂HPO₄, 0.75 g/L KH₂PO₄, 0.7 g/L MgSO₄·7H₂O, 0.017 g/L MnSO₄·5H₂O, 0.01 g/L FeSO₄·7H₂O, 2 g/L (NH₄)₂SO₄, 1 g/L NaCl, 2 g/L asparagine, 0.004 g/L p-aminobenzoic acid, 5 g/L yeast extract, and 10 g/L glucose) under anaerobic conditions until OD600=1.0. A culture solution was left on ice for 10 minutes, and then cultured cells were centrifuged at 7000 g for 10 minutes at 4° C. After washing a cell pellet three times with a buffer solution, the cells were suspended in 2 ml of the same buffer solution, thereby preparing cells for transformation. Then, 5.0 μg of the pCK2-MCS shuttle plasmid was added to 500 ul of the cells for transformation as prepared. Then, the mixture was subjected to electroporation (4 mm cuvette, 2.5 kV, 25 uF) using a Gene pulser II from a Bio-Rad company. Then, a transformed strain was identified in a medium having chloramphenicol antibiotic added thereto.

Before the electroporation, all the plasmids used for the transformation was methylated in Escherichia coli TOP10 strain transformed with pAN1 plasmid (which has a gene that methylates internal cytosine when a GCNGC sequence is present) so that all the plasmids was not affected by a restriction system of the Clostridium acetobutylicum strain.

Production of Transformant of pLK1-MCS Shuttle Plasmid

A transformant of the pLK1-MCS shuttle plasmid was produced in the same manner as the production method of the transformant of the pCK2-MCS shuttle plasmid, except that the pLK1-MCS shuttle plasmid instead of the pCK2-MCS shuttle plasmid was introduced, and erythromycin antibiotic instead of chloramphenicol was added to a medium.

<3-3> Evaluation of Replication Stability in Clostridium acetobutylicum

A replication stability of the shuttle plasmid pCK2-MCS plasmid was evaluated based on Clostridium acetobutylicum strain including the pCK2-MCS shuttle plasmid prepared in <3-2>. The evaluation method employed a previously reported method (Shin MH, Jung MW, Lee J-H, Kim MD, Kim KH. 2008. Strategies for producing recombinant sucrose phosphorylase originating from Bifidobacterium longum in Escherichia coli JM109. Process Biochemistry 43: 822-828).

First, the shuttle plasmid was introduced into the Clostridium acetobutylicum ATCC 824 strain via electroporation and the strain was cultured at 37° C. for 2 days in anaerobic culture conditions in a solid medium including chloramphenicol antibiotic. Thereafter, one colony was cultured in a culture tube containing 40 ml CGM liquid medium at 37° C. without antibiotics until a cell concentration was 1.0 (OD600 nm). In this connection, the cell concentration was measured using a spectrophotometer (Hach, USA).

The cultured cells were diluted and then were smeared onto and cultured in a solid CGM medium without antibiotics and at 37° C. for 36 hours, and thereafter, the number of colonies as formed was identified. Thereafter, 50 colonies as formed were replica-plated onto a CGM solid medium including chloramphenicol antibiotic, and then the number of cells having shuttle plasmid loss were identified. When the shuttle plasmid is lost in the cell, the cell has no chloramphenicol antibiotic resistance. Thus, the colony may not be formed in the replica plating.

Further, 40 uL of an initial liquid culture solution was again inoculated into a 40 ml CGM liquid without antibiotics (was diluted to have 1/1000 of an initial culture solution concentration) and, then, the process as described above was repeated 10 times. The number of cells having the loss of the shuttle plasmid was identified. Thus, loss stability of the shuttle plasmid for 100 generations was evaluated. It was assumed that each generation was divided 10 times because the inoculation occurred in a state in which the cell concentration was diluted to have 1/1000 of an initial culture solution concentration each time. (210-1024).

As a result, it was identified that the pCK2-MCS plasmid had excellent replication stability and improved loss stability, compared to pMTL500E (which includes a pAMβ1 replication origin and a nucleic acid sequence for encoding a replication protein) which has been used as a conventional Escherichia coli-Clostridium shuttle plasmid, the pLK1-MCS shuttle plasmid, as well as the pGS1-MCS shuttle plasmid (Table 6).

TABLE 6

| Plasmid | Number of colonies | Temperature(° C.) |
|---|---|---|
| pCK2-MCS | 3.9 × 10³ | 37 |
| pLK1-MCS | 4.3 × 10² | 37 |
| pMTL500E | 2.6 × 10² | 37 |
| pGS1-MCS | 4.6 × 10² | 37 |

<3-4> Evaluation of Replication Stability in Escherichia coli

The fact that the shuttle plasmid pCK2-MCS may be stably replicated has been confirmed based on the Experimental Example <3-2> indicating that the pCK2-MCS was transformed and methylated in the Escherichia coli TOP10 including the pAN1 plasmid to methylate the shuttle plasmid pCK2-MCS (Table 7).

TABLE 7

| Plasmid | Number of colonies | Temperature(° C.) |
|---|---|---|
| pCK2-MCS | 3.8 × 10⁶ | 37 |
| pLK1-MCS | 2.3 × 10⁶ | 37 |

<3-5> Replication Convenience

The pLK1-MCS shuttle plasmid includes a first antibiotic resistant gene expressed in Escherichia coli and capable of being selectively labeled in Escherichia coli, and a second antibiotic resistant gene expressed in Clostridium and capable of being selectively labeled in Clostridium. When removing one of the antibiotic resistant genes to reduce the size of the shuttle plasmid, the pLK1-MCS shuttle plasmid may not be applied to both of Clostridium and Escherichia coli.

To the contrary, the pCK2-MCS shuttle plasmid according to the present disclosure may use an antibiotic resistant gene expressed in both Escherichia coli and Clostridium and capable of being selectively labeled in both Escherichia coli and Clostridium. Therefore, the pCK2-MCS shuttle plasmid according to the present disclosure may have a smaller size and may be applied to both Clostridium and Escherichia coli.

Therefore, it was identified that the replication convenience of the pCK2-MCS shuttle plasmid according to the present disclosure was greater. Further, the pCK2-MCS shuttle plasmid has the same replication origin, that is, the replication origin site of the pUC19 plasmid, as that of the pLK1-MCS shuttle plasmid. Thus, when the pCK2-MCS shuttle plasmid and the pLK1-MCS shuttle plasmid are transformed together, the pCK2-MCS shuttle plasmid and the pLK1-MCS shuttle plasmid may be expressed to the same degree.

### <Experimental Example 4> Production of Recombinant Strain

<4-1> Preparation of Clostridium acetobutylicum TM2-1-C

Clostridium acetobutylicum TM2-1-C(Accession No. KCTC 12604BP) was used herein. Clostridium acetobutylicum TM2-1-C is a strain obtained by treating Clostridium acetobutylicum ATCC824 Δpta Δbuk with methyl-N-nitro-N-nitrosoguanidine (NTG) to cause a random mutagenic. Clostridium acetobutylicum TM2-1-C has simultaneous fermentation performance of glucose and xylose.

For reference, the Clostridium acetobutylicum ATCC824 Δpta Δbuk is a recombinant strain obtained by applying a method described in WO2011/037415 to a Clostridium acetobutylicum ATCC824 wild-type strain. The Clostridium acetobutylicum ATCC824 Δpta Δbuk is a recombinant microorganism in which a pta as a gene for expressing phosphotransacetylase, and a buk as a gene for expressing butyrate kinase are simultaneously deleted in the Clostridium acetobutylicum ATCC824 wild type strain.

<4-2> Production of Recombinant Strain TM-pLK1-AdhE1-CtfAB

Production of plasmid vector pLK1-AdhE1-CtfAB

3 genes of AdhE1, CtfA and CtfB were cloned into the pLK1-MCS plasmid vector, thereby to produce a plasmid vector pLK1-AdhE1-CtfAB including an artificial operon.

A specific production method thereof is as follows. An AdhE1 gene (SEQ ID NO: 20) of Clostridium acetobutylicum was amplified via PCR reaction using a primer pair (SEQ ID NO: 21 and 22). The amplified fragment was ligated with fragments obtained by cleaving the pLK1-MCS shuttle plasmid using PmeI/XhoI, thereby preparing pLK1-AdhE1 (Table 8).

TABLE 8

| SEQ ID NO | ATGAAAGTCACAACAGTAAAGGAATTAGATGAAAAACTCAAGGTAATTAAAGAAGCTCAAAAAAAA |
|---|---|
| 20 | TTCTCTTGTTACTCGCAAGAAATGGTTGATGAAATCTTTAGAAATGCAGCAATGGCAGCAATCGACG |
| | CAAGGATAGAGCTAGCAAAAGCAGCTGTTTTGGAAACCGGTATGGGCTTAGTTGAAGACAAGGTTA |
| | TAAAAAATCATTTTGCAGGCGAATACATCTATAACAAATATAAGGATGAAAAAACCTGCGGTATAATT |
| | GAACGAAATGAACCCTACGGAATTACAAAAATAGCAGAACCTATAGGAGTTGTAGCTGCTATAATCC |
| | CTGTAACAAACCCCACATCAACAACAATATTTAAATCCTTAATATCCCTTAAAACTAGAAATGGAATTT |
| | TCTTTTCGCCTCACCCAAGGGCAAAAAAATCCACAATACTAGCAGCTAAAACAATACTTGATGCAGC |
| | CGTTAAGAGTGGTGCCCCGGAAAATATAATAGGTTGGATAGATGAACCTTCAATTGAACTAACTCAAT |
| | ATTTAATGCAAAAAGCAGATATAACCCTTGCAACTGGTGGTCCCTCACTAGTTAAATCTGCTTATTCTT |
| | CCGGAAAACCAGCAATAGGTGTTGGTCCGGGTAACACCCCAGTAATAATTGATGAATCTGCTCATATA |
| | AAAATGGCAGTAAGTTCAATTATATTATCCAAAACCTATGATAATGGTGTTATATGTGCTTCTGAACAAT |
| | CTGTAATAGTCTTAAAATCCATATATAACAAGGTAAAAGATGAGTTCCAAGAAAGAGGAGCTTATATA |
| | ATAAAGAAAAACGAATTGGATAAAGTCCGTGAAGTGATTTTTAAAGATGGATCCGTAAACCCTAAAA |
| | TAGTCGGACAGTCAGCTTATACTATAGCAGCTATGGCTGGCATAAAAGTACCTAAAACCACAAGAAT |
| | ATTAATAGGAGAAGTTACCTCCTTAGGTGAAGAAGAACCTTTTGCCCACGAAAAACTATCTCCTGTT |
| | TTGGCTATGTATGAGGCTGACAATTTTGATGATGCTTTAAAAAAAGCAGTAACTCTAATAAACTTAGG |
| | AGGCCTCGGCCATACCTCAGGAATATATGCAGATGAAATAAAAGCACGAGATAAAATAGATAGATTT |
| | AGTAGTGCCATGAAAACCGTAAGAACCTTTGTAAATATCCCAACCTCACAAGGTGCAAGTGGAGATC |
| | TATATAATTTTAGAATACCACCTTCTTTCACGCTTGGCTGCGGATTTTGGGGAGGAAATTCTGTTTCCG |
| | AGAATGTTGGTCCAAAACATCTTTTGAATATTAAAACCGTAGCTGAAAGGAGAGAAAACATGCTTTG |
| | GTTTAGAGTTCCACATAAAGTATATTTTAAGTTCGGTTGTCTTCAATTTGCTTTAAAAGATTTAAAAGA |
| | TCTAAAGAAAAAAAGAGCCTTTATAGTTACTGATAGTGACCCCTATAATTTAAACTATGTTGATTCAAT |
| | AATAAAAATACTTGAGCACCTAGATATTGATTTTAAAGTATTTAATAAGGTTGGAAGAGAAGCTGATC |
| | TTAAAACCATAAAAAAAGCAACTGAAGAAATGTCCTCCTTTATGCCAGACACTATAATAGCTTTAGGT |
| | GGTACCCCTGAAATGAGCTCTGCAAAGCTAATGTGGGTACTATATGAACATCCAGAAGTAAAATTTG |
| | AAGATCTTGCAATAAAATTTATGGACATAAGAAAGAGAATATATACTTTCCCAAAACTCGGTAAAAA |
| | GGCTATGTTAGTTGCAATTACAACTTCTGCTGGTTCCGGTTCTGAGGTTACTCCTTTTGCTTTAGTAAC |
| | TGACAATAACACTGGAAATAAGTACATGTTAGCAGATTATGAAATGACACCAAATATGGCAATTGTAG |
| | ATGCAGAACTTATGATGAAAATGCCAAAGGGATTAACCGCTTATTCAGGTATAGATGCACTAGTAAAT |
| | AGTATAGAAGCATACACATCCGTATATGCTTCAGAATACACAAACGGACTAGCACTAGAGGCAATAC |
| | GATTAATATTTAAATATTTGCCTGAGGCTTACAAAAACGGAAGAACCAATGAAAAAGCAAGAGAGA |
| | AAATGGCTCACGCTTCAACTATGGCAGGTATGGCATCCGCTAATGCATTTCTAGGTCTATGTCATTCCA |
| | TGGCAATAAAATTAAGTTCAGAACACAATATTCCTAGTGGCATTGCCAATGCATTACTAATAGAAGAA |
| | GTAATAAAATTTAACGCAGTTGATAATCCTGTAAAACAAGCCCCTTGCCCACAATATAAGTATCCAAA |
| | CACCATATTTAGATATGCTCGAATTGCAGATTATATAAAGCTTGGAGGAAATACTGATGAGGAAAAG |
| | GTAGATCTCTTAATTAACAAAATACATGAACTAAAAAAAGCTTTAAATATACCAACTTCAATAAAGGA |
| | TGCAGGTGTTTTGGAGGAAAACTTCTATTCCTCCCTTGATAGAATATCTGAACTTGCACTAGATGATC |
| | AATGCACAGGCGCTAATCCTAGATTTCCTCTTACAAGTGAGATAAAAGAAATGTATATAAATTGTTTTA |
| | AAAAACAACCTTAA |
| SEQ ID NO | CACGTTTAAACATGAAAGTCACAACAGTAAAGGAATTAGAT |
| 21 | |
| SEQ ID NO | CACACTCGAGTTAAGGNTGTTTTTTAAAACAATTTATATACA |
| 22 | |

Thereafter, the Thiolase promoter (SEQ ID NO: 23) of Clostridium acetobutylicum was amplified via PCR using a primer pair (SEQ ID NO: 24 and 25). The amplified fragment was ligated with fragments obtained by cleaving the pLK1-AdhE1 with XhoI/BglII, thereby preparing pLK1-AdhE1-pThl-MCS.

Thereafter, a CtfAB gene group (SEQ ID NO: 26) was amplified via PCR using a primer pair (SEQ ID NO: 27 and 28). The amplified fragment was ligated with fragments obtained by cleaving the pLK1-AdhE1-pThl-MCS using BglII/EcoRI. Thus, a plasmid vector pLK1-AdhE1-CtfAB was prepared (Table 9).

TABLE 91

| | |
|---|---|
| SEQ ID NO 23 | TTAGAATGAAGTTTCTTATGCACAAGTATTTTTTATTACATTAATATAGTTAAAATATAAACTT |
| | ATGTATTTATGCTAAAACATGATTTTAAGGGGGTTAGCATATGCATAAGTTTAATTTTTTTGTT |
| | AAAAAATATTAAACTTTGTGTTTTTTTTAACAAAATATATTGATAAAAATAATAATAGTGGGT |
| | ATAATTAAGTTGTTAGAGAAAACGTATAAATTAGGGATAAACTATGGAACTTATGAAATAGA |
| | TTGAAATGGTTTATCTGTTACCCCGTATCAAAATTT |
| SEQ ID NO 24 | CACACTCGAGTTAGAATGAAGTTTCTTATGCACAAGTATTTTTTA |
| SEQ ID NO 25 | TCTAGATCTTAACCTCCTAAATTTTGATACGGGGTAACAG |
| SEQ ID NO 26 | ATGAACTCTAAAATAATTAGATTTGAAAATTTAAGGTCATTCTTTAAAGATGGGATGACAATT |
| | ATGATTGGAGGTTTTTTAAACTGTGGCACTCCAACCAAATTAATTGATTTTTTAGTTAATTTAA |
| | ATATAAAGAATTTAACGATTATAAGTAATGATACATGTTATCCTAATACAGGTATTGGTAAGT |
| | TAATATCAAATAATCAAGTAAAAAAGCTTATTGCTTCATATATAGGCAGCAACCCAGATACT |
| | GGCAAAAAACTTTTTAATAATGAACTTGAAGTAGAGCTCTCTCCCCAAGGAACTCTAGTGGA |
| | AAGAATACGTGCAGGCGGATCTGGCTTAGGTGGTGTACTAACTAAAACAGGTTTAGGAACT |
| | TTGATTGAAAAAGGAAAGAAAAAAATATCTATAAATGGAACGGAATATTTGTTAGAGCTAC |
| | CTCTTACAGCCGATGTAGCATTAATTAAAGGTAGTATTGTAGATGAGGCCGGAAACACCTTC |
| | TATAAAGGTACTACTAAAAACTTTAATCCCTATATGGCAATGGCAGCTAAAACCGTAATAGT |
| | TGAAGCTGAAAATTTAGTTAGCTGTGAAAAACTAGAAAAGGAAAAAGCAATGACCCCCGG |
| | AGTTCTTATAAATTATATAGTAAAGGAGCCTGCATAAAATGATTAATGATAAAAACCTAGCG |
| | AAAGAAATAATAGCCAAAAGAGTTGCAAGAGAATTAAAAAATGGTCAACTTGTAAACTTAG |
| | GTGTAGGTCTTCCTACCATGGTTGCAGATTATATACCAAAAAATTTCAAAATTACTTTCCAAT |
| | CAGAAAACGGAATAGTTGGAATGGGCGCTAGTCCTAAAATAAATGAGGCAGATAAAGATG |
| | TAGTAAATGCAGGAGGAGACTATACAACAGTACTTCCTGACGGCACATTTTTCGATAGCTCA |
| | GTTTCGTTTTCACTAATCCGTGGTGGTCACGTAGATGTTACTGTTTTAGGGGCTCTCCAGGTA |
| | GATGAAAAGGGTAATATAGCCAATTGGATTGTTCCTGGAAAAATGCTCTCTGGTATGGGTG |
| | GAGCTATGGATTTAGTAAATGGAGCTAAGAAAGTAATAATTGCAATGAGACATACAAATAA |
| | AGGTCAACCTAAAATTTTAAAAAAATGTACACTTCCCCTCACGGCAAAGTCTCAAGCAAATC |
| | TAATTGTAACAGAACTTGGAGTAATTGAGGTTATTAATGATGGTTTACTTCTCACTGAAATTA |
| | ATAAAAACACAACCATTGATGAAATAAGGTCTTTAACTGCTGCAGATTTACTCATATCCAAT |
| | GAACTTAGACCCATGGCTGTTTAA |
| SEQ ID NO 27 | CACAGATCTATGAACTCTAAAATAATTAGATTTGAAAATTTAAG |
| SEQ ID NO 28 | CACGAATTCTTAAACAGCCATGGGTCTAAGTTCATTGGATATGA |

Production of Recombinant Strain TM-pLK1-AdhE1-CtfAB

A plasmid vector pLK1-AdhE1-CtfAB was introduced into Clostridium acetobutylicum TM2-1-C. This was conducted in the same manner as in the transformation method of the above <3-2>. As a result, a recombinant strain TM-pLK1-AdhE1-CtfAB into which AdhE1, CtfA and CtfB genes were introduced was produced.

<4-3> Production of Recombinant Strain TM-pLK1-AdhE1-CtfAB+pCK2-Xylba

Production of Plasmid Vector pCK2-Xy1BA

A native operon portion including a gene XylB for encoding xylose kinase as an xylose metabolic enzyme and a gene XylA for encoding xylulose isomerase was cloned into the pCK2-MCS plasmid vector. Thus, a plasmid vector pCK2-XylBA including XylBA operon was produced. A specific production method thereof is as follows. An operon (SEQ ID NO: 29) including the genes for encoding xylose kinase and xylulose isomerase of the Clostridium acetobutylicum was amplified via PCR using a primer pair (SEQ ID NO: 30 and 31). The amplified fragment was ligated with fragments obtained by cleaving pCK2-MCS with PstI/NotI, thereby preparing pCK2-XylBA. The plasmid vector pCK2-XylBA produced in this way was not transformed into TM2-1-C strain but was continuously transformed into the TM-pLK1-AdhE1-CtfAB strain having pLK1-AdhE1-CtfAB introduced thereto (Table 10).

TABLE 10

| | |
|---|---|
| SEQ ID NO 29 | ATGAGGTATTTATTAGGTATAGACGTTGGAACATCAGGAACCAAGACAGCTTTGTTTGATGAA |
| | TGTGGAAATACAATAAAAACTTCAACGCATGAGTATGAGTTATTTCAACCACAGGTTGGATGG |
| | GCAGAACAAAATCCTGAAAATTGGTGGACAGCTTGTGTTAAAGGTATAAGAGAAGTAATTGA |
| | AAAAAGTAAAATTGATCCTTTAGATATTAAAGGAATTGGTATAAGTGGACAAATGCATGGACT |
| | TGTTTTGATTGACAAAGAGTACAAAGTAATTAGAAACTCCATAATTTGGTGTGATCAGAGAAC |
| | AGAAAAGGAATGTACTCAGATTACAGATACCATAGGAAAAGAAAAATTAATTAGGATAACAG |
| | GCAATCCTGCATTAACGGGTTTCACCCTTTCAAAACTATTATGGGTTAGGAATAATGAACCAG |
| | ACAATTATAAGAGAATATATAAGGTTCTTCTTCCTAAGGATTATATAAGATTTAAACTTACTGG |
| | AGTTTTTGCTGCCGAAGTTTCAGATGCTAGTGGTACTCAGATGTTAGATATAAATACTAGAAAT |
| | TGGAGTGAAGAACTTTTAGATGACTTAAGAATAGATAAGAACATATTACCTGATGTATATGAA |
| | TCAGTTGTTGTTAGTGGATGCGTTATAGAAAAAGCTTCAAAAGAGACTAAGTTAGCAGTTAAC |
| | ACTCCCGTTGTAGGTGGGGCAGGAGATCAAGCAGCTGGAGCTATAGGGAATGGAATCGTTA |
| | GAGAAGGTTTAATATCAACAGTGATAGGAACTTCTGGAGTAGTATTTGCGGCTACAGATACG |
| | CCTAGATTTGATAGTAAGGGAAGAGTTCATACTCTTTGTCATGCAGTGCCTAATAAGTGGCAT |
| | ATAATGGGAGTTACTCAGGGAGCAGGCTTATCATTAAATTGGTTTAAAAGGACATTTTGTGCA |
| | AAAGAAATTTTAGAAAGTAAAGAGGCTGGAATTAATATTTATGATTTATTGACCGAAAAAGCA |
| | TCACAATCAAAGCCTGGTTCTAATGGGATAATTTATTTGCCATACCTTATGGGTGAAAGAACA |
| | CCGCACATAGATCCAAATGTAAAAGGAGCTTTTTTAGGTATATCACTTATAAATAACCACAAT |
| | GATTTTGTGCGTAGTATATTAGAAGGAGTAGGCTTTAGTTTAAAGAATTGTCTCGATATTATTG |
| | AGAATATGAAGGTTAATATTGAGGAAATACGAGTAAGTGGTGGAGGTGCAGAAAGCAGCAT |
| | ATGGAGACAAATATTGTCAGATATATTTAACTATGAGCTTACAACAGTAAAGGCATCAGAAGG |
| | ACCAGCACTTGGCGTAGCAATACTTGCAGGAGTTGGTGCAGGAATATATAATTCCGTGGAAG |
| | AGGCITGTGACAAAATAGTAAAAGGAAACGAAAAGGTTATGCCAAATGCAAATTTAATAGAA |
| | GTATATTCAAAGGTATATGAGGTATATAATTCAGCTTATCCTAAAATAAAAGATATATAAATAG |
| | TTAAAAGTAGGAGCGCTTTTATTTAAAGAGTAGATTATCTAAAAAACTTATTAAACAAACTTTA |
| | AAAACATATATTTATTAGAGAAGTTTAAATCATATTGGTAAGATATTTTTTAAATAAAACCAATT |
| | TAAATTATATGGGGAGTGTGAGAATATGGTAATTAAAAGTGTTACAGACAAGGAGTTCAAAA |
| | AATATGGCAAGATATTAAGAGATTATGATTGTACAGAGATAATAGAAAAAATGAAATCTACAC |
| | CACTTCCAGGTGATGTAGTATATGAACCATCAATTAAAGAACTTGAAGAGCTTGATATTGCAA |
| | AGGAATTAAAGGAAAGAGAATATGGAGAGCTTCCAATACAAGTAGGATACTGCAATGGTAAC |
| | AATTATTTATTAAATGCAGTCGAATATCATCGTTCATCTGAAATAAATATAGCAGTAACAGATT |
| | TCATACTACTTTTAGGCTCTAGAGATGATATTGAAGAAGATTACTCATATAGTACTTCTAAAAT |
| | TAAAGCATTTAAAGTTCCAGCTGGTACTGCTATTGAGGTTTATGCAACTACTCTTCACTATGCA |
| | CCTTGCAATGTAAATGAAGGGGGATTCAGATGTGTTGTGGTTTTGCCAAGAGATACAAATTTG |
| | CCTTTAGAAAAAGAGTATAAAAAAACTGGTGAAGATGCTCTACTTTTTGCTAAAAACAAATGG |
| | TTAATTGCCCATAAGGATACTAACCTAGATAAAGAAGGAGCATTTATAGGATTAATAGGTAAA |
| | AATATTTCAATAAAATAAAATGGAGGAATTAAAATGAATAATACACCAAAATTAAAATTAGGA |
| | ATTGTTGCAGTAAGTAGAGACTGCTTTCCAATGGAATTATCAGAGAATAGAAGAAAGGCAGT |
| | AGTAGATGCTTACAATGGAGAAATCTTCGAGTGCTTAACAACTGTTGAAAATGAAAAAGATAT |
| | GAGAAAAGCTTTAAAAGAAGTGAAAAGTGCAGGTGTAAATGCACTAGTAGTATATTTAGGTA |
| | ACTTTGGACCAGAAACTCCAGAAACTTTAATTGCTAAAGAATTTGATGGACCAGTTATGTTTG |
| | CAGCCGCAGCAGAAGAGAGAGGAGACAATCTAGTTAATGGACGTGGAGATGCTTATTGTGG |
| | AATGCTAAATGCAAGCTATAATTTAGCACTTAGAAACATAAATGCTTATATTCCTGAATATCCA |
| | GTGGGAACAGCTTCAGATGTTGCAAATATGATTAATGAATTTGTACCGGTTGCTACTGCATTA |
| | ATTGGATTAAAGAATTTAAAGATTATTTCTTTTGGGCCAAGACCTCAGGATTTCCTAGCTTGCA |
| | ATGCTCCTATTAAGCAGTTATATAATTTAGGTGTTGAAATAGAGGAAAATTCGGAGCTTGATTT |
| | GTTTGCCTCATTTAATGAACATGAAAACGATTCTAGAATTCCAGATGTAGTAAAAGACATGGA |
| | GGAAGAGTTAGGTGAGGGCAATAAAATGCCTGGTATTTTGCCAAAGCTTGCTCAATATGAAC |
| | TTACATTACTAGATTGGGCAGAGGAGCATAAAGGATCAAGAGAATATGTTGTGTTTGCAAATA |
| | AATGCTGGCCAGCGTTTCAAACACAATTTGGATGTGTGCCTTGCTATGTAAACAGTAGACTAA |
| | CGGCTAGAGGAATTCCAGTATCTTGTGAGGTTGACATTTATGGAGCTTTAAGTGAATACATTG |
| | GAACATGTGTAAGTCAAGATGTTGTAACCTTACTTGATATTAATAACACAGTACCAAAAGATA |
| | TGTATGAATCAGAAATTAAAAGCAAATTTAACTATACGTTAAAGGATACCTTCATGGGTTTTCA |
| | CTGTGGAAACACAGCAGCATGTAAATTAACAAGCGGAACTATGAAAAATCAAATGATTATGG |
| | CAAGAGCTCTTGAGCCAAATCAAGAGCCTAATATAACTAGAGGAACGTTAGAGGGAGATATT |
| | GTACCAGGAGAAATCACTTTCTTCCGTCTACAAAGTAATGCAGATAGTGAGTTAACAGCCTAT |
| | GTAGCAGAAGGAGAGGTTTTACCTGTTAAGACACGCTCTTTTGGATCTATTGGAGTATTTGCA |
| | ATTCCTCAAATGGGGAGATTTTACCGCCATGTACTAATTGAAAATAGATTCCCACACCATGGT |
| | GCAGTTGCATTTGGACATTTTGGTAAAGCAATTTATAATTTGTTTAGATATTTGGGAGTTAAAG |
| | AGGTAGGTTTTAATAGGCCAAAGGAAATGCTATATAAAACAGAAAATCCTTTTGAGTAA |
| SEQ ID NO 30 | CGCTGCAGATGAGGTATTTATTAGGTATAGACGTTG |
| SEQ ID NO 31 | GGCGGCCGCTTACTCAAAAGGATTTTCTGTTTTATATAGC |

Production of Recombinant Strain TM-pLK1-AdhE1-CtfAB+pCK2-XylBA

The plasmid vector pCK2-Xy1BA was introduced into the recombinant strain TM-pLK1-AdhE1-CtfAB as produced in the above <4-2>. As a result, a recombinant strain TM-pLK1-AdhE1-CtfAB+pCK2-XylBA into which XylB and XylA genes were introduced was produced. The recombinant strain TM-pLK1-AdhE1-CtfAB+pCK2-XylBA (Clostridium sp. XyloMaxl) was deposited with the Korea Research Institute of Bioscience and Biotechnology on May 16, 2017 (Accession number: KCTC13268BP).

<4-4> Production of Recombinant Strain TM-pLK1-AdhE1-CtfAB+pCK2-Xylba

Production of Recombinant Strain TM-pCK2-XylBA

The plasmid vector pCK2-Xy1BA produced in the above <4-3> was first introduced into a Clostridium acetobutylicum TM2-1-C strain using the transformation method of the above <3-2>, thereby first producing a TM-pCK2-Xy1BA recombinant strain.

Production of Recombinant Strain TM-pLK1-AdhE1-CtfAB+pCK2-XylBA

The plasmid vector pLK1-AdhE1-CtfAB was introduced into the recombinant strain TM-pCK2-XylBA as first produced above. In this connection, the plasmid vector pLK1-AdhE1-CtfAB was introduced into the recombinant strain TM-pCK2-XylBA as first produced above in the same manner in the production method of the recombinant strain TM-pLK1-AdhE1-CtfAB as described in the above <4-2>. As a result, a recombinant strain TM-pLK1-AdhE1-CtfAB+pCK2-XylBA as in the above <4-3> was produced.

That is, unlike the above <4-3> in which the plasmid vector pLK1-AdhE1-CtfAB was introduced and, thereafter, the plasmid vector pCK2-XylBA was introduced, in this <4-4>, the plasmid vector pCK2-XylBA was introduced and then the plasmid vector pLK1-AdhE1-CtfAB was introduced. Thus, the same recombinant strain as in the above <4-3> was produced.

<4-5> Production of Recombinant Strain TM-pLK1-AdhE1-CtfAB+pCK2-Xylba

Unlike <4-3> and <4-4> in which the different plasmid vectors were sequentially, a method of simultaneously inserting the two different plasmid vectors into a TM2-1-C strain was used. 5.0 ug of pLK1-AdhE1-CtfAB plasmid vector and pCK2-XylBA plasmid vector was added to 500 ul of cells for transformation. Then, electroporation was performed. Then, a transformed strain was identified in a medium including both erythromycin antibiotic and chloramphenicol antibiotic. In this connection, the transformation method is the same as the transformation method in the above <3-2>.

### <Experimental Example 5> Production of Bio-Butanol Using Batch Culture

An ability with which the recombinant microorganisms produces butanol was tested using a batch culture method. The recombinant microorganism employed the recombinant strain TM-pLK1-AdhE1-CtfAB produced in the Experimental Example <4-2>, and the recombinant strain TM-pLK1-AdhE1-CtfAB+pCK2-XylBA produced in the Experimental Example <4-3>. A wild type C. acetobutylicum ATCC824 was used as a control.

Each of the microorganisms was smeared on a CGM solid medium, and was subjected to anaerobic culture at 37° C. overnight. Each cultured colony was inoculated into a 50 ml disposable tube (Falcon, USA) containing 40 ml of CCM, and was allowed to be left at 37° C. and was subjected to the anaerobic culture until OD600=1. The seed culture was again inoculated into a CGM liquid medium containing 400 ml 6% glucose, and was left at 37° C. and was subjected to anaerobic culture until OD600=1. Then, the culture was added to a fermenter vessel containing 1.6 L of CGM liquid medium with 8% glucose added thereto and was cultured therein. pH was maintained to be 5.0 using ammonium hydroxide (NH₄OH) during the anaerobic culture. The anaerobic condition was maintained while nitrogen was injected thereto at a rate of 20 ml/min A concentration of each of butanol and a mixed solvent generated every 3 hours after start of the glucose based culture was analyzed. Gas chromatography (Agilent, USA) was used for the analysis of butanol and the mixed solvent, and the analysis conditions are shown in Table 8 below. For analysis of a concentration of each of sugar and organic acid, the culture was centrifuged, and then a supernatant was obtained, and, then, the concentration of each of sugar and organic acid was identified using HPLC and a sugar analyzer. The HPLC condition was as follows: water containing 0.01 N sulfuric acid was used as a mobile phase and a flow rate was 0.6 ml/min. A column was embodied as Aminex87H and Aminex87P (Bio-Rad, USA). The resulting sugar and organic acid were analyzed using a RI (Reflective Index) detector.

As a result, it was identified that the recombinant strain Clostridium acetobutylicum TM-pLK1-AdhE1-CtfAB+pCK2-XylBA has high productivity and selectivity of butanol and high pentose consumption in a sugar condition with high pentose content (Table 11).

TABLE 11

| | | C. acetobutylicum | C. acetobutylicum |
|---|---|---|---|
| | C. acetobutylicum | TM-pLK1-AdhE1- | TM-pLK1-AdhE1- |
| | ATCC824 | CtfAB | CtfAB + pCK2-XylBA |
| acetone (g/L) | 0.7 | 0.6 | 1.3 |
| ethanol (g/L) | 0.5 | 0.9 | 1.4 |
| butanol (g/L) | 4.0 | 12.9 | 14.7 |
| total ABE (g/L) | 5.2 | 14.4 | 17.4 |
| ethanol selectivity (%) | 8.8 | 6.3 | 8.2 |
| butanol selectivity (%) | 77.2 | 89.5 | 84.5 |
| ABE productivity (g/L/h)* | 0.19 | 0.87 | 0.83 |
| cumulative C5 consumption | 5.8 | 16.7 | 23.3 |
| percentage (%) | | | |
| ABE yield (%) | 11.5 | 27.5 | 30.8 |
| acetone selectivity (%) | 14.0 | 4.2 | 7.3 |
| Acetic acid (g/L) | 5.4 | 2.4 | 2.6 |
| Butyric acid (g/L) | 7.1 | 2.5 | 1.0 |

### <Experimental Example 6> Production of Bio-Butanol Using Fed-Batch Culture

An ability with which the recombinant microorganisms produces butanol was tested using a fed-batch culture method. The recombinant microorganism employed the recombinant strain TM-pLK1-AdhE1-CtfAB produced in the Experimental Example <4-2> and the recombinant strain TM-pLK1-AdhE1-CtfAB+pCK2-XylBA produced in the Experimental Example <4-3>. The wild type C. acetobutylicum ATCC824 was used as a control.

Each of the microorganisms was smeared on a CGM solid medium, and was subjected to anaerobic culture at 37° C. overnight. Each cultured colony was inoculated into a 50 ml disposable tube (Falcon, USA) containing 40 ml of CCM, and was allowed to be left at 37° C. and was subjected to the anaerobic culture until OD600=1. The seed culture was again inoculated into a CGM liquid medium containing 400 ml 6% glucose, and was left at 37° C. and was subjected to anaerobic culture until OD600=1. Then, the culture was added to a fermenter vessel containing 1.6 L of CGM liquid medium having 8% glucose and 200 g of an absorbing agent for selectively absorbing butanol added thereto, and was cultured therein. pH was maintained to be 5.0 using ammonium hydroxide (NH₄OH) during the anaerobic culture. The anaerobic condition was maintained while nitrogen was injected thereto at a rate of 20 ml/min. A concentration of each of butanol and a mixed solvent generated every 3 hours after start of the glucose based culture was analyzed. In order to maintain the glucose concentration in the culture medium at a level equal to or higher than 10 g/L, a 700 g/L glucose solution was used as a feeding solution.

To produce butanol at high yield, high productivity and high selectivity using a continuous culture, it is very important to keep an ethanol concentration low so that ethanol does not cause toxicity to the strain during the culture time. From the results of the fed-batch culture, it was identified that the recombinant strain Clostridium acetobutylicum TM-pLK1-AdhE1-CtfAB+pCK2-XylBA maintained high butanol-producing performance while maintaining low ethanol selectivity. Thus, the recombinant strain Clostridium acetobutylicum TM-pLK1-AdhE1-CtfAB+pCK2-XylBA was expected to be suitable for a long-term continuous culture (Table 12).

TABLE 12

| | | C. acetobutylicum | C. acetobutylicum |
|---|---|---|---|
| | C. acetobutylicum | TM-pLK1-AdhE1- | TM-pLK1-AdhE1- |
| | ATCC824 | CtfAB | CtfAB + pCK2-XylBA |
| acetone (g/L) | 0.4 | 2.6 | 2.5 |
| ethanol (g/L) | 0.3 | 2.0 | 2.0 |
| butanol (g/L) | 2.6 | 26.4 | 25.7 |
| total ABE (g/L) | 3.3 | 30.9 | 30.2 |
| ethanol selectivity (%) | 9.0 | 6.3 | 6.6 |
| butanol selectivity (%) | 78.9 | 85.2 | 85.1 |
| ABE productivity (g/L/h)* | 0.11 | 1.21 | 0.96 |
| cumulative C5 consumption | 1.5 | 22.4 | 27.5 |
| percentage (%) | | | |
| ABE yield (%) | 5.6 | 31.9 | 31.8 |
| acetone selectivity (%) | 12.1 | 8.5 | 8.3 |
| Acetic acid (g/L) | 6.7 | 2.4 | 2.4 |
| Butyric acid (g/L) | 12.5 | 1.0 | 1.1 |

### <Experimental Example 7> Production of Bio-Butanol Using Continuous Culture Method

An ability with which the recombinant microorganisms produces butanol was tested using a continuous culture method. The recombinant microorganism employed the recombinant strain TM-pLK1-AdhE1-CtfAB produced in the Experimental Example <4-2>, and the recombinant strain TM-pLK1-AdhE1-CtfAB+pCK2-XylBA produced in the Experimental Example <4-3>. The wild type C. acetobutylicum ATCC824 was used as a control.

First, an incubator for the continuous culture process was prepared based on a disclosure in Korean patent application No. 10-2012-0038770. First, in order to prevent the adsorption agent from eluting up and down in the column having a volume of 3 L and thus being lost, a filter of about 150 um was installed, and then a stirrer was installed, and 200 g of the adsorbent was filled therein, thereby completing two columns. The columns were connected to the incubator using a silicone tube, and then, a pump was mounted, such that the culture solution circulated between the two columns. Each of an inlet and an outlet of the column was equipped with a 4-way valve. Thus, when the adsorbent in the column was saturated with the butanol and mixed solvent during the culturing process, a solvent for elution was flowed into the column to allow removal of the column in real time. When removing the first column, the culture was circulated to the second column, so that the flow of the culture was kept continuous. The circulating direction of the culture medium was a direction from the top to the bottom of the column, but the direction does not matter. The microorganisms were cultured in the prepared incubator. First, 800 ml of the seed culture subjected to the anaerobic culture process in the CGM liquid medium overnight was added to a reactor containing 3.2 L of CGM liquid medium. In this way, the culturing process started. In this Experimental Example, the seed culture was cultured using a general batch fermentation. When, after the incubation start, a butanol concentration reached about 7 to 8 g/L, the culture medium passed through the column at a flow rate of 50 ml/min under control of the pump and was circulated. As the culture medium passed through the column, the adsorbent was suspended in the culture medium to form a dilute slurry phase. Thus, flow of the culture medium was not blocked due to a cell flock but passed through the column. The culture medium samples immediately before and after the passage through the column were collected. Then, the butanol concentration was controlled to be kept 8 g/L or lower.

As a result, it was identified that the recombinant strain Clostridium acetobutylicum TM-pLK1-AdhE1-CtfAB+pCK2-XylBA has high butanol productivity and selectivity when using EFB (Empty fruit Bunch) hydrolysate with high pentose content. Further, it was identified that the recombinant strain Clostridium acetobutylicum TM-pLK1-AdhE1-CtfAB+pCK2-XylBA has higher pentose consumption. This effect occurred equally in both a test using a EFB simulant which simulated a sugar concentration and a composition of the EFB hydrolysate, and a test using an actual EFB hydrolysate (Table 13 and Table 14).

TABLE 13

| | C. acetobutylicum | C. acetobutylicum |
|---|---|---|
| | TM-pLK1- | TM-pLK1-AdhE1- |
| | AdhE1-CtfAB | CtfAB + pCK2-XylBA |
| acetone (g/L) | 120.0 | 125.6 |
| ethanol (g/L) | 115.3 | 124.1 |
| butanol (g/L) | 852.5 | 943.0 |
| total ABE (g/L) | 1087.7 | 1192.7 |
| ethanol selectivity (%) | 10.6 | 10.4 |
| butanol selectivity (%) | 78.4 | 79.1 |
| ABE productivity (g/L/h)* | 3.22 | 3.45 |
| cumulative C5 consumption | 32.2 | 37.9 |
| percentage (%) | | |
| culture duration (h) | 85 | 85 |
| ABE yield (%) | 32.8 | 34.5 |
| acetone selectivity (%) | 11.0 | 10.5 |
| Acetic acid (g/L) | 70.6 | 56.8 |
| Butyric acid (g/L) | 29.4 | 38.6 |

Use of EFB simulant (85 hour fermentation result)

TABLE 14

| | C. acetobutylicum | C. acetobutylicum |
|---|---|---|
| | TM-pLK1- | TM-pLK1-AdhE1- |
| | AdhE1-CtfAB | CtfAB + pCK2-XylBA |
| acetone (g/L) | 90.5 | 130.6 |
| ethanol (g/L) | 97.4 | 127.9 |
| butanol (g/L) | 718.4 | 896.7 |
| total ABE (g/L) | 906.8 | 1155.1 |
| ethanol selectivity (%) | 10.7 | 11.1 |
| butanol selectivity (%) | 79.2 | 77.6 |
| ABE productivity (g/L/h)* | 2.37 | 3.07 |
| cumulative C5 consumption | 34.5 | 36.0 |
| percentage (%) | | |
| culture duration (h) | 95 | 95 |
| ABE yield (%) | 33.8 | 34.1 |
| acetone selectivity (%) | 10.0 | 11.3 |
| Acetic acid (g/L) | 66.4 | 64.8 |
| Butyric acid (g/L) | 47.0 | 43.4 |

Use of actual EFB hydrolysate (95 hour fermentation result)

<Deposition information>

Depository name: Korea Research Institute of Bioscience and Biotechnology

Accession number: KCTC12604BP

Date of accession: 20140610

Depository name: Korea Research Institute of Bioscience and Biotechnology

Accession number: KCTC12755BP

Date of Deposit: 20150204

Depository name: Korea Research Institute of Bioscience and Biotechnology

Accession number: KCTC13268BP

Date of Deposit: 20170516

Depository name: Korea Research Institute of Bioscience and Biotechnology

Accession number: KCTC13267BP

Date of Deposit: 20170516

### INDUSTRIAL AVAILABILITY

The present disclosure relates to a shuttle plasmid replicable in Clostridium and Escherichia coli, the shuttle plasmid including: a nucleic acid sequence of a first replication origin replicable in Escherichia coli; a nucleic acid sequence for encoding a replication protein region derived from a pUB110 plasmid; and an expression terminator sequence of a gene.

### SEQUENCE LISTING FREE TEXT

SEQ ID NO: 1 is a sequence of a primer to amplify a replication origin of a pUB110 plasmid and a replication protein coding region.

SEQ ID NO: 2 is a sequence of a primer to amplify a replication origin of a pUB110 plasmid and a replication protein coding region.

SEQ ID NO: 3 is a nucleic acid sequence of a replication origin of a pUB110 plasmid.

SEQ ID NO: 4 is a nucleic acid sequence for encoding a replication protein (RepA).

SEQ ID NO: 5 is an amino acid sequence of a replication protein (RepA).

SEQ ID NO: 6 is a sequence of DNA including a promoter and a multiple cloning site.

SEQ ID NO: 7 is a nucleic acid sequence of an erythromycin resistant gene.

SEQ ID NO: 8 is a nucleic acid sequence of an ampicillin resistant gene.

SEQ ID NO: 9 is a nucleic acid sequence of a replication origin of a pUC19 plasmid.

SEQ ID NO: 10 is a nucleic acid sequence of a plasmid pLK1-MCS.

SEQ ID NO: 11 is a primer sequence used for amplification of a replication origin site of a pUC19 plasmid of a pLK1-MCS plasmid.

SEQ ID NO: 12 is a primer sequence used for amplification of a replication origin site of a pUC19 plasmid of a pLK1-MCS plasmid.

SEQ ID NO: 13 is a nucleic acid sequence of a thiolase promoter region.

SEQ ID NO: 14 is a nucleic acid sequence of a DNA fragment obtained by amplifying a replication origin site of a pUC19 plasmid, a thiloase promoter region, a MCS site, a replication origin site of a pUB110 plasmid, and a replication protein coding region of a pLK1-MCS plasmid.

SEQ ID NO: 15 is a sequence of primers used to amplify a chloramphenicol resistant gene included in a pGS1-MCS plasmid.

SEQ ID NO: 16 is a sequence of primers used to amplify a chloramphenicol resistant gene included in a pGS1-MCS plasmid.

SEQ ID NO: 17 is a nucleic acid sequence of a DNA fragment obtained by amplifying a chloramphenicol resistant gene included in a pGS1-MCS plasmid.

SEQ ID NO: 18 is a DNA sequence including a transcription terminator of a acetoacetate decarboxylase gene.

SEQ ID NO: 19 is a nucleic acid sequence of a pCK2-MCS shuttle plasmid.

SEQ ID NO: 20 is a nucleic acid sequence of an AdhE1 gene of Clostridium acetobutylicum.

SEQ ID NO: 21 is a primer sequence used to amplify an AdhE 1 gene of Clostridium acetobutylicum.

SEQ ID NO: 22 is a sequence of primers used to amplify an AdhE 1 gene of Clostridium acetobutylicum.

SEQ ID NO: 23 is a sequence of a thiolase promoter of Clostridium

acetobutylicum.

SEQ ID NO: 24 is a primer sequence used to amplify a sequence of a

Thiolase promoter of Clostridium acetobutylicum.

SEQ ID NO: 25 is a primer sequence used to amplify a sequence of a Thiolase promoter of Clostridium acetobutylicum.

SEQ ID NO: 26 is a nucleic acid sequence of a CtfAB gene group.

SEQ ID NO: 27 is a sequence of a primer used to amplify a CtfAB gene group.

SEQ ID NO: 28 is a sequence of a primer used to amplify a CtfAB gene group.

SEQ ID NO: 29 is a nucleic acid sequence of an operon including genes for encoding xylose kinase and xylulose isomerase of Clostridium acetobutylicum.

SEQ ID NO: 30 is a sequence of a primer used to amplify an operon including genes for encoding xylose kinase and xylulose isomerase of Clostridium acetobutylicum.

SEQ ID NO: 31 is a sequence of a primer used to amplify an operon including genes for encoding xylose kinase and xylulose isomerase of Clostridium acetobutylicum.

1. A shuttle plasmid replicable in Clostridium and Escherichia coli, the shuttle plasmid including:
a nucleic acid sequence of a first replication origin replicable in Escherichia coli;
a nucleic acid sequence for encoding a replication protein region derived from a pUB110 plasmid; and
an expression terminator sequence of a gene.

2. The shuttle plasmid of claim 1, wherein the shuttle plasmid further includes an antibiotic resistant gene expressed in Clostridium and Escherichia coli.

3. The shuttle plasmid of claim 2, wherein the antibiotic resistant gene is a chloramphenicol resistant gene.

4. The shuttle plasmid of claim 1, wherein the shuttle plasmid has a size of 3000 bp to 4000 bp.

5. The shuttle plasmid of claim 1, wherein the expression terminator sequence is a nucleic acid sequence of a transcription terminator of a gene for encoding acetoacetate decarboxylase.

6. The shuttle plasmid of claim 1, wherein the shuttle plasmid is replicable in both Clostridium and Escherichia coli without replacing an antibiotic resistant gene.

7. The shuttle plasmid of claim 1, wherein the shuttle plasmid further includes a following i) or a following ii):
i) a nucleic acid sequence of a thiolase promoter region and a multiple cloning site (MCS),
ii) a nucleic acid sequence of a replication origin of a pUB110 plasmid.

8. A method for producing a recombinant microorganism, the method including:
preparing the shuttle plasmid according to claim 1;
introducing at least one gene into the shuttle plasmid, thereby producing a first recombinant shuttle plasmid; and
introducing the first recombinant shuttle plasmid into a microorganism.

9. The method of claim 8, wherein the at least one gene is at least one selected from a group consisting of a gene for encoding xylose kinase and a gene for encoding xylulose isomerase.

10. The method of claim 8, wherein the microorganism has an acetyl coenzyme A biosynthetic pathway and a butyryl coenzyme A biosynthetic pathway.

11. The method of claim 8, wherein the microorganism includes a gene for encoding alcohol/aldehyde dehydrogenase and a gene for encoding coenzyme A transferase.

12. The method of claim 8, wherein the gene introduced into the shuttle plasmid is at least one selected from a group consisting of a gene for encoding xylose kinase, a gene for encoding xylulose isomerase, a gene for encoding alcohol/aldehyde dehydrogenase, and a gene for encoding coenzyme A transferase.

13. The method of claim 8, wherein the method further includes, after introducing the first recombinant shuttle plasmid into the microorganism, introducing a second recombinant shuttle plasmid into the microorganism.

14. The method of claim 13, wherein the second recombinant shuttle plasmid includes the same gene as the gene introduced into the first recombinant shuttle plasmid, or a gene different from the gene introduced into the first recombinant shuttle plasmid.

15. The method of claim 13, wherein the second recombinant shuttle plasmid includes at least one gene selected from a group consisting of a gene for encoding xylose kinase, a gene for encoding xylulose isomerase, a gene for encoding alcohol/aldehyde dehydrogenase, and a gene for encoding coenzyme A transferase,
wherein the second recombinant shuttle plasmid includes the same gene as the gene introduced into the first recombinant shuttle plasmid, or a gene different from the gene introduced into the first recombinant shuttle plasmid.

16. The method of claim 8, wherein the microorganism is a recombinant microorganism which is prepared by
introducing a second recombinant shuttle plasmid into a microorganism.

17. The method of claim 16, wherein the gene introduced into the first recombinant shuttle plasmid is at least one selected from a group consisting of a gene for encoding xylose kinase, a gene for encoding xylulose isomerase, a gene for encoding alcohol/aldehyde dehydrogenase, and a gene for encoding coenzyme A transferase.

18. The method of claim 16, wherein the second recombinant shuttle plasmid includes at least one gene selected from a group consisting of a gene for encoding xylose kinase, a gene for encoding xylulose isomerase, a gene for encoding alcohol/aldehyde dehydrogenase, and a gene for encoding coenzyme A transferase.

19. The method of claim 8, wherein the step of introducing the first recombinant shuttle plasmid into a microorganism is preformed by
simultaneously introducing the first recombinant shuttle plasmid and a second recombinant shuttle plasmid into a microorganism.

20. (canceled)

21. (canceled)

22. A method for obtaining a fermentation product, the method including:
culturing the recombinant microorganism produced by the production method of claim 8, thereby producing a culture; and
obtaining a fermentation product from the culture.

23. (canceled)

24. (canceled)

25. (canceled)

26. (canceled)

27. (canceled)

28. (canceled)
